(19) United States
(12) Patent Application Publication
(10) Pub. No.: US 2025/0082280 A1
(43) Pub. Date: Mar. 13, 2025

(54) METHOD FOR TRANSFERRING A PRESSURE WAVE FROM PERIPHERAL SITE TO CENTRAL SITE

(71) Applicant: CALIFORNIA INSTITUTE OF TECHNOLOGY, Pasadena, CA (US)

(72) Inventors: Alessio TAMBORINI, PASADENA, CA (US); Morteza GHARIB, ALTADENA, CA (US); Arian AGHILINEJAD, LOS ANGELES, CA (US)

(21) Appl. No.: 18/829,089

(22) Filed: Sep. 9, 2024

Related U.S. Application Data
(60) Provisional application No. 63/658,734, filed on Jun. 11, 2024.
(60) Provisional application No. 63/620,041, filed on Jan. 11, 2024.
(60) Provisional application No. 63/607,430, filed on Dec. 7, 2023.
(60) Provisional application No. 63/537,274, filed on Sep. 8, 2023.

(51) Int. Cl. A61B 5/00; A61B 5/02; A61B 5/021; A61B 5/024; A61B 5/025

(52) U.S. Cl. CPC A61B 5/7275; A61B 5/02007; A61B 5/02125; A61B 5/02416; A61B 5/025; A61B 5/7257; A61B 5/7264

(57) ABSTRACT

The disclosure relates to techniques for transferring a pulse pressure waveform signal of a peripheral vascular site to a central vascular site. In some implementations, a method includes: obtaining a time-domain representation of a first pulse pressure waveform signal of a brachial artery of a subject; converting the time-domain representation of the first pulse pressure waveform signal to a frequency-domain representation of the first pulse pressure waveform signal; selecting a first plurality of frequency components of the frequency-domain representation of the first pulse pressure waveform signal; predicting, using a non-linear mapping, based on the first plurality of frequency components, a second plurality of frequency components of a frequency-domain representation of a second pulse pressure waveform signal of an ascending aorta or left ventricle of the subject; and converting the frequency-domain representation of the second pulse pressure waveform signal to a time-domain representation of the second pulse pressure waveform signal.

### CROSS-REFERENCE TO RELATED APPLICATIONS

This application claims the benefit of U.S. Provisional Patent Application No. 63/658,734, filed Jun. 11, 2024, and titled “A Method for Generating Non-Invasive Patient-Specific Pressure-Volume Loops at the Left Ventricular Site”, U.S. Provisional Patent Application No. 63/620,041 filed Jan. 11, 2024, and titled “An Implementation of the Method for Transferring a Pressure Wave from Peripheral Site Central Site for the Reconstruction of the Left Ventricular Wave”, U.S. Provisional Patent Application No. 63/607,430, filed Dec. 7, 2023, and titled “An Implementation of the Method for Transferring a Pressure Wave From Peripheral Site Central Site for the Reconstruction of the Left Ventricular Wave”, and U.S. Provisional Patent Application No. 63/537,274, filed Sep. 8, 2023, and titled “A Method for Transferring a Pressure Wave from Peripheral Site to Central Site”. All of the above applications are incorporated herein by reference in their entirety.

### BACKGROUND

The cardiac pulse waveform and its morphological characteristics are recognized as valuable sources of information for evaluating the cardiovascular state. The cardiac pulse waveform is formed by the superposition of forward and reflected waves within the arterial system. Structural properties, inclusive of geometric and material properties, and functional properties of the cardiovascular system modify the shape of these propagating waves. Aging and disease generate distinct modifications to the cardiovascular system that are consequently reflected in the propagating pressure waves. As such, the pressure waveform contains information regarding the structural and functional properties of the cardiovascular system.

Cardiac pulse waveforms, peripheral or central, allow physicians to indirectly infer heart health from a pressure-time signal. In recent years, a variety of new medical technologies have been developed to assess cardiac function and health using a peripheral pressure measurement. For example, the radial pressure waveform is often preferred due to its lower variability between examiners, as well as its greater comfort for patients. Moreover, measuring the radial pressure pulse is of great interest due to its accessibility in wrist-worn wearable devices.

While peripheral waveforms are more accessible, central waveforms have been shown to be more informative and lead to better patient outcome predictions. Given the invasive nature of central pulse waveform measurement, reconstruction of central waveforms given a peripheral measurement has been a topic of great interest in non-invasive cardiology for many decades. To this end, several methods have been proposed to transfer peripheral waveforms to central waveforms. To assess the central pressure and hemodynamics using peripheral-based measurements, there is an essential need to transfer the peripheral measurement to the central estimation. The generalized transfer function (GTF) is the most widely adopted transfer function among the existing methods to estimate the central pressure waveform from the peripheral measurement and is considered as the gold standard approach. Use of this transfer function for radial-based measurements has been well-established clinically. To obtain the GTF, multiple individual transfer functions are averaged. However, this procedure introduces inaccuracy due to the non-negligible variability in the physiological parameters between subjects. Additionally, previous studies reported poor generalizability for this method when applied to a new group of subjects based on the priorly generated GTF.

### SUMMARY

The technology described herein relates to techniques for transferring a pulse pressure waveform signal of a peripheral vascular site of a subject to a central vascular site of the subject.

In one embodiment, a method comprises: obtaining a time-domain representation of a first pulse pressure waveform signal of a brachial artery of a subject; converting the time-domain representation of the first pulse pressure waveform signal to a frequency-domain representation of the first pulse pressure waveform signal; selecting a first plurality of frequency components of the frequency-domain representation of the first pulse pressure waveform signal; predicting, using a non-linear mapping, based on the first plurality of frequency components, a second plurality of frequency components of a frequency-domain representation of a second pulse pressure waveform signal of an ascending aorta or a left ventricle (LV) of the subject; and converting the frequency-domain representation of the second pulse pressure waveform signal to a time-domain representation of the second pulse pressure waveform signal.

In some implementations, predicting the second plurality of frequency components comprises predicting each of the second plurality of frequency components using at least some of the first plurality of frequency components as an input to the non-linear mapping.

In some implementations, a number of the first plurality of frequency components is different from a number of the second plurality of frequency components.

In some implementations, converting the time-domain representation of the first pulse pressure waveform signal to the frequency-domain representation of the first pulse pressure waveform signal comprises: applying a fast Fourier transform (FFT) to the time-domain representation of the first pulse pressure waveform signal; selecting the first plurality of frequency components of the frequency-domain representation of the first pulse pressure waveform signal comprises selecting a first number of Fourier harmonic modes of the frequency-domain representation of the first pulse pressure waveform signal; and converting the frequency-domain representation of the second pulse pressure waveform signal to the time-domain representation of the second pulse pressure waveform signal comprises: applying an inverse FFT to the frequency-domain representation of the second pulse pressure waveform signal.

In some implementations, the non-linear mapping comprises a regressor; and predicting the second plurality of frequency components comprises: applying the regressor to the first number of Fourier harmonic modes to obtain a second number of Fourier harmonic modes of the frequency-domain representation of the second pulse pressure waveform signal.

In some implementations, the first number of Fourier harmonic modes is 5 or more, and the second number of Fourier harmonic modes is 10 or more.

In some implementations, the second pulse pressure waveform signal is of the LV of the subject.

In some implementations, the non-linear mapping comprises a trained model; and predicting the second plurality of frequency components comprises: predicting, using the trained model, given at least the first plurality of frequency components as an input, the second plurality of frequency components.

In some implementations, the method further comprises calculating one or more waveform parameters corresponding to the time-domain representation of the first pulse pressure waveform signal; and predicting the second plurality of frequency components comprises: predicting, using the trained model, given at least the first plurality of frequency components and the one or more waveform parameters as inputs, the second plurality of frequency components.

In some implementations, the method further comprises obtaining one or more physiological parameters of the subject, the one or more physiological parameters comprising an age, weight, height, or gender of the subject; and predicting the second plurality of frequency components comprises: predicting, using the trained model, given at least the first plurality of frequency components and the one or more physiological parameters as inputs, the second plurality of frequency components.

In some implementations, obtaining the time-domain representation of the first pulse pressure waveform signal comprises: performing, using a blood pressure (BP) cuff of a BP cuff system, a blood pressure measurement of the subject to obtain one or more blood pressure values corresponding to the subject; inflating, based on the one or more blood pressure values corresponding to the subject, the BP cuff to a subject specific pressure value; and capturing, using the BP cuff system, while the BP cuff is inflated to the subject specific pressure value, the first pulse pressure waveform signal associated with the brachial artery of the subject.

In some implementations, the one or more blood pressure values comprise a systolic blood pressure (SBP) of the subject; and the subject specific pressure value comprises a supra systolic blood pressure (sSBP) greater than the SBP.

In some implementations, the first pulse pressure waveform signal corresponds to a single cardiac cycle; and capturing the first pulse pressure waveform signal comprises: capturing, using the BP cuff system, while the BP cuff is inflated to the subject specific pressure value, a third pulse pressure waveform signal corresponding to more than one cardiac cycle; and isolating the first pulse pressure waveform signal from the third pulse pressure waveform signal.

In some implementations, the method further comprises: applying pulse waveform analysis (PWA) to the time-domain representation of the second pulse pressure waveform signal to assess a cardiovascular health condition of the subject.

In some implementations, the second pulse pressure waveform signal corresponds to a central aortic pressure of the subject; and applying PWA to the time-domain representation of the second pulse pressure waveform signal to assess the cardiovascular health condition of the subject comprises: estimating, based one or more parameters of the time-domain representation of the second pulse pressure waveform signal, an arterial stiffness or a subendocardial viability ratio.

In some implementations, the second pulse pressure waveform signal corresponds to a left ventricular pressure of the subject; and applying pulse waveform analysis to the time-domain representation of the second pulse pressure waveform signal to assess the cardiovascular health condition of the subject comprises: estimating, based one or more parameters of the time-domain representation of the second pulse pressure waveform signal, a ventricular contractility or left ventricular end diastolic pressure.

In some implementations, the second pulse pressure waveform signal is of the LV of the subject, and the method further comprises: obtaining an LV pressure curve of the subject associated with the time-domain representation of the second pulse pressure waveform signal; capturing, using an imaging device, one or more images of the LV of the subject; measuring, using the one or more images of the LV, an LV volume curve of the subject; and obtaining, using the LV pressure curve and the LV volume curve, an LV pressure-volume curve.

In one embodiment, a non-transitory computer-readable medium has executable instructions stored thereon that, when executed by a processor, cause the processor to perform operations comprising: obtaining a time-domain representation of a first pulse pressure waveform signal of a brachial artery of a subject; converting the time-domain representation of the first pulse pressure waveform signal to a frequency-domain representation of the first pulse pressure waveform signal; selecting a first plurality of frequency components of the frequency-domain representation of the first pulse pressure waveform signal; predicting, using a non-linear mapping, based on the first plurality of frequency components, a second plurality of frequency components of a frequency-domain representation of a second pulse pressure waveform signal of an ascending aorta or a left ventricle of the subject; and converting the frequency-domain representation of the second pulse pressure waveform signal to a time-domain representation of the second pulse pressure waveform signal.

In one embodiment, a method comprises: obtaining, using a brachial cuff, a time-domain representation of a first pulse pressure waveform signal of a brachial artery of a subject; reconstructing, using a frequency-domain representation of the first pulse pressure waveform signal, a frequency-domain representation of a second pulse pressure waveform signal of a LV of the subject; obtaining, using the frequency-domain representation of the second pulse pressure waveform signal of the LV of the subject, an LV pressure curve of the subject in the time domain; capturing, using an imaging device, one or more images of the LV of the subject; measuring, using the one or more images of the LV, an LV volume curve of the subject; and obtaining, using the LV pressure curve and the LV volume curve, an LV pressure-volume curve.

In some implementations, obtaining the time-domain representation of the first pulse pressure waveform signal of the brachial artery of the subject comprises: capturing, using the brachial cuff, multiple blood pressure measurements of the subject; and the multiple blood pressure measurements and the one or more images of the LV are synchronously captured.

In some implementations, the method further comprises capturing an electrocardiogram (ECG) of the subject; and obtaining the LV pressure-volume curve comprises synchronizing, using the ECG, the LV pressure curve and the LV volume curve.

Other features and aspects of the disclosed technology will become apparent from the following detailed description, taken in conjunction with the accompanying drawings, which illustrate, by way of example, the features in accordance with implementations of the disclosed technology. The summary is not intended to limit the scope of any inventions described herein, which are defined by the claims and equivalents.

### BRIEF DESCRIPTION OF THE DRAWINGS

The present disclosure, in accordance with one or more implementations, is described in detail with reference to the following figures. The figures are provided for purposes of illustration only and merely depict example implementations. Furthermore, it should be noted that for clarity and ease of illustration, the elements in the figures have not necessarily been drawn to scale.

FIG. 1A is a high level diagram depicting an example system within which the technology described herein can be implemented to provide central pulse waveform reconstruction, in accordance with some implementations of the disclosure.

FIG. 1B illustrates one particular embodiment in which a pulse pressure waveform signal measured for a brachial artery of a subject is directly measured using a BP cuff system, and reconstructed into an estimated pulse pressure waveform signal for a LV of the subject, using the system of FIG. 1A.

FIG. 2 is an operational flow diagram illustrating an example method that can be implemented to reconstruct an estimated pulse pressure waveform associated with a central vascular site of subject, given a measured pulse pressure waveform associated with a peripheral vascular site of the subject, in accordance with some implementations of the disclosure.

FIG. 3 is an operational flow diagram illustrating an example method that leverages physiological boosting to reconstruct an estimated pulse pressure waveform associated with a central vascular site of subject, in accordance with some implementations of the disclosure.

FIG. 4 is an operational flow diagram illustrating an example method that performs PWA of an estimated pulse pressure waveform associated with a central vascular site of subject, to measure a patient cardiovascular condition, in accordance with some implementations of the disclosure.

FIG. 5 is an operational flow diagram illustrating one example method for the reconstruction of a non-invasive LV PV loop of a subject, in accordance with some implementations of the disclosure.

FIG. 6A depicts one example of an LV pressure curve and LV volume curve that can be generated using the techniques described with reference to FIG. 5.

FIG. 6B depicts one example of an LV pressure-volume curve that can be generated from the LV pressure curve and LV volume curve of FIG. 6A, using the techniques described with reference to FIG. 5.

FIG. 7A is a diagram illustrating a brachial site of a subject where a pulse pressure waveform measurement can be captured using a BP cuff system, and the central aortic site for which the pulse pressure waveform can be reconstructed for the subject, in accordance with some implementations of the disclosure.

FIG. 7B shows a predicted pulse pressure waveform at an ascending aorta reconstructed using a measured brachial pulse pressure waveform, in accordance with some implementations of the disclosure, and the actual pulse pressure waveform measurement captured using an invasive catheter placed in the ascending aorta.

FIG. 7C include plots showing the accuracy of pulse waveform parameters measured using PWA on a reconstructed waveform as illustrated by FIG. 7B when compared to those measured on the actual waveform measured with a catheter in the ascending aorta.

FIG. 7D include plots showing the accuracy of pulse waveform parameters measured using PWA on a reconstructed waveform as illustrated by FIG. 7B when compared to those measured on the actual waveform measured with a catheter in the ascending aorta.

FIG. 8A is a diagram illustrating a brachial site of a subject where a pulse pressure waveform measurement can be captured using a BP cuff system, and the LV site for which the pulse pressure waveform can be reconstructed for the subject, in accordance with some implementations of the disclosure.

FIG. 8B includes a comparison plot showing true (measured) values for left ventricular maximum pressure, versus predicted values for left ventricular maximum pressure predicted using techniques in accordance with the disclosure.

FIG. 8C includes a first plot showing an example of reconstructed and measured left ventricular pressure waveform for a single cardiac cycle (top), and a second plot showing an example of reconstructed and measured left ventricular pressure waveforms for multiple sequential cardiac cycles representing a continuous measurement modality (bottom).

FIG. 9A includes plots showing a ten second segment of a continuous pulse waveform measurements obtained for two subjects using a catheter (TRUE), versus the reconstructed pulse waveforms using the GTF and using Fourier-based machine learning methods in accordance with the disclosure.

FIG. 9B includes plots showing a ten second segment of a continuous pulse waveform measurements obtained for a subject using a catheter (TRUE), versus the reconstructed pulse waveforms using the GTF and using Fourier-based machine learning methods in accordance with the disclosure.

FIG. 10A are plots showing the normalized Root-Mean-Squared-Error (nRMSE) between the measured left ventricular waveform and the self-reconstructed waveform based on a different number of Fourier modes, in accordance with some implementations of the disclosure.

FIG. 10B includes three examples of plots showing the comparison between the measured and reconstructed left ventricular pulse waveforms (left panel of each figure) and the first derivative (right panel of each figure) utilizing a Fourier-based machine learning approach, in accordance with some implementations of the disclosure.

FIG. 10C includes three examples of plots showing the comparison between the measured and reconstructed left ventricular pulse waveforms (left panel of each figure) and the first derivative (right panel of each figure) utilizing a Fourier-based machine learning approach, in accordance with some implementations of the disclosure.

FIG. 10D illustrates a comparison of the results from three different model Fourier-based machine learning (F-ML) structures inclusive of a Base F-ML model, a F-ML model with principal component analysis to reduce dimensionality, and a F-ML model with physiological boosting.

FIG. 11 is a diagram showing an example of a blood pressure cuff system, in accordance with some implementations of the disclosure.

FIG. 12A is a diagram showing an example of a resistive component having a fixed orifice, in accordance with some implementations of the disclosure.

FIG. 12B is a diagram showing an example of a resistive component having an in-line filter, in accordance with some implementations of the disclosure.

FIG. 12C is a diagram showing an example of a resistive component having a reduced internal diameter, in accordance with some implementations of the disclosure.

FIG. 12D is a schematic of the capacitive component including an elastic tube, in accordance with some implementations of the disclosure.

FIG. 12E is a schematic of the capacitive component including tube with a piston cylinder, in accordance with some implementations of the disclosure.

FIG. 13 is a flow diagram showing an example of a pulse pressure waveform measurement method, in accordance with some implementations of the disclosure.

FIG. 14A is an example plotting of a left ventricle pressure-volume (“PV”) loop in a young patient, in accordance with some implementations of the disclosure.

FIG. 14B is an example plotting of a left ventricle pressure-volume (“PV”) loop in an old patient, in accordance with some implementations of the disclosure.

FIG. 15 is a flow diagram showing an example of a left ventricular end-diastolic pressure risk prediction method, in accordance with some implementations of the disclosure.

FIG. 16 is an example plotting of an envelope function reconstructed at a specified pressure level and three pressure holds, in accordance with some implementations of the disclosure.

FIG. 17 is an example plotting of an envelope function to estimate SBP and DBP changes following pulse amplitude fluctuations, in accordance with some implementations of the disclosure.

The figures are not exhaustive and do not limit the present disclosure to the precise form disclosed.

### DETAILED DESCRIPTION

The pulse pressure waveform encodes information regarding the state of health of an individual, and the waveform has predictive value in detecting cardiovascular disease. However, direct measurement of the pulse pressure waveform within a central vascular site such as the left ventricle typically requires invasive catheterization, which is expensive and carries associated risk. As such, there is a need for improved techniques for obtaining a central pulse pressure waveform without direct measurement. To this end, the technology described herein is directed to novel techniques for transferring a pulse pressure waveform signal of a peripheral vascular site of a subject to a central vascular site of the subject.

The reconstruction techniques described herein are based on the wave propagation and reflection that occurs in the cardiovascular system. A pressure wave occurring with a peripheral vascular site such as the brachial artery generates a pressure-time signal that can be directly measured by, for example, using a BP cuff system further described below. The information carried in these waves is well captured in the harmonic content. By applying a non-linear mapping (e.g., a trained model) to the wave harmonic content of the peripheral vascular site in the frequency space, the wave harmonic content of the central vascular site in the frequency space can be accurately predicted. As further described below, time-frequency analytical tools can be used to dimensionally reduce the input parameter space to a non-linear mapping for predicting the harmonic content of the central waveform. The predicted harmonics can be subsequently converted to the time domain to yield the pulse pressure waveform signal corresponding to the central vascular site.

Particular implementations described herein are directed to reconstructing the pulse pressure waveform signal of an ascending aorta or left ventricle of a subject given the measured pulse pressure waveform of the brachial artery of the subject. For example, some techniques described herein are directed to transferring a pressure wave from a peripheral vascular site to the left ventricular pressure waveform by applying a regressor on the wave harmonic content in the frequency space.

Additional implementations of the disclosure are directed to improving waveform reconstruction accuracy by leveraging physiological information of the subject. In accordance with such implementations, one or more waveform parameters of the peripheral pressure waveform signal and/or one or more known physiological parameters of the subject can be considered when applying the non-linear mapping to predict wave harmonic content of the central vascular site.

Further implementations of the disclosure are directed to using the reconstructed waveform to evaluate the cardiovascular health of the subject. In accordance with such implementations, quantitative parameters of the reconstructed central pulse pressure waveform can be extracted via PWA to evaluate the cardiovascular health of the subject.

Yet further implementations of the disclosure are directed to using the reconstructed waveform to generate a patent-specific pressure-volume (PV) loop of the LV. In accordance with such implementations, non-invasive imaging modalities can be used to obtain an LV volume curve, and a brachial cuff can be used to obtain the reconstructed LV pressure curve. Thereafter the LV PV loop can be derived, and the ventricular function and other heart health parameters can be assessed by examining the PV loop.

Various advantages can be realized by implementing the technology described herein. The technology described herein provides methods and systems for mapping peripheral vascular measurements, which are easy and non-invasive to obtain, to a central pressure waveform that is of clinical and practical interest. As further described herein, testing of the techniques described herein for reconstructing the central pressure waveform generates waveform predictions for central pressure with superior accuracy compared with current standard methods such as methods that utilize the GTF. By virtue of the superior accuracy of the reconstructed central waveform, parameters extracted by applying PWA on the reconstructed waveform can have improved accuracy, improving the quality of non-invasive diagnosis. Further still, the techniques described herein enable reconstruction of the left ventricular waveform given a peripheral measurement. Moreover, the techniques described herein enable generation of a fully non-invasive and truly patient-specific PV loop of the LV. These and other advantages that can be realized by implementing the technology described herein are further exemplified by the description that follows.

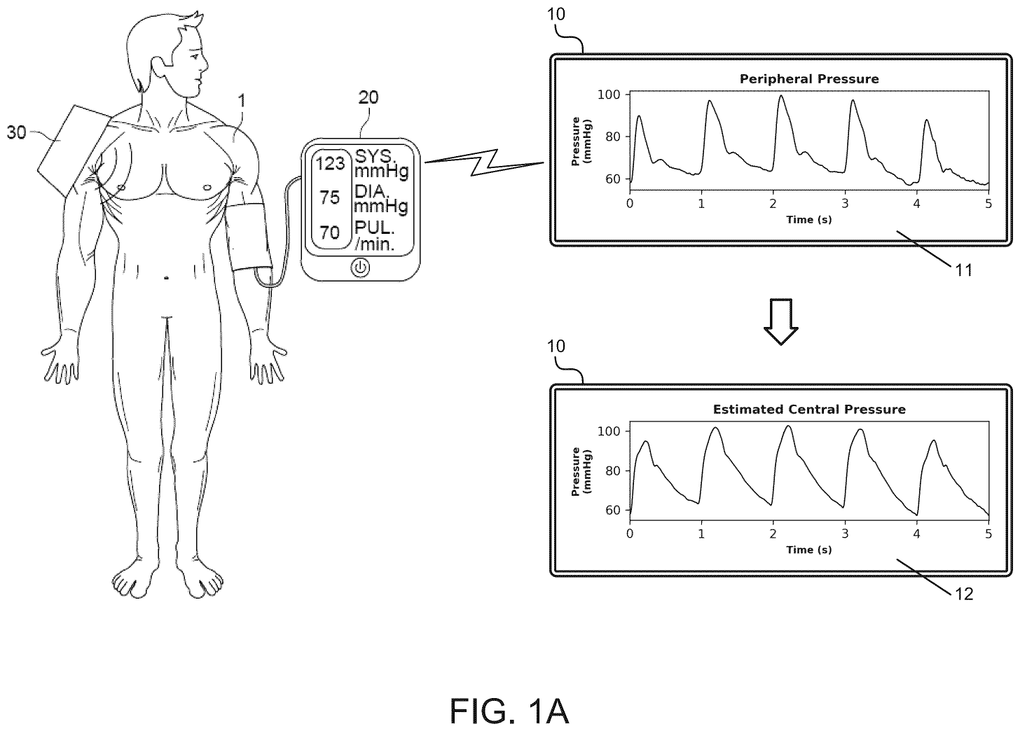

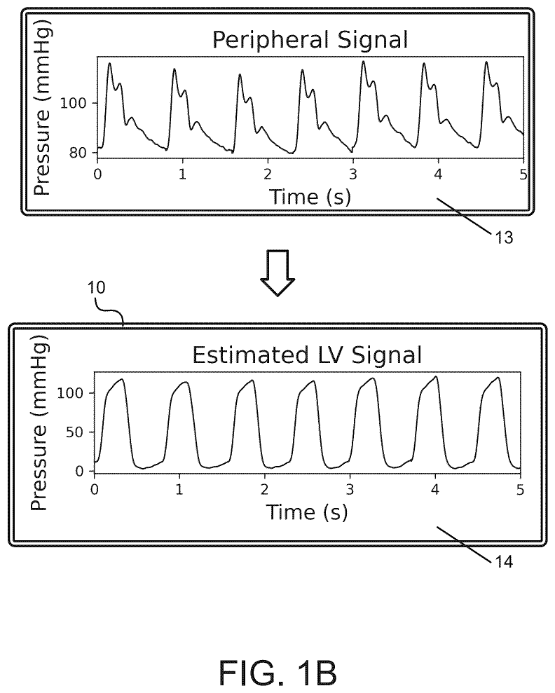

FIG. 1A is a high level diagram depicting an example system within which the technology described herein can be implemented to provide central pulse waveform reconstruction. As depicted, the system includes a central pulse waveform reconstruction device 10 in communication with a BP cuff system 20 worn by a patient/subject 1. During operation, the BP cuff system 20 is configured to individually, or in combination with waveform reconstruction device 10, capture a peripheral vascular site pulse pressure waveform signal 11 representing the high-frequency vibrations that propagate as pressure waves within the arterial system of subject 1. In this example, the BP cuff system 20 can capture the pulse pressure waveform associated with a brachial artery of subject 1. It should be appreciated, however, that in other implementations the pulse pressure waveform of some other peripheral vascular site of subject 1 can be captured using BP cuff system 20 or some other suitable pulse pressure waveform capture device.

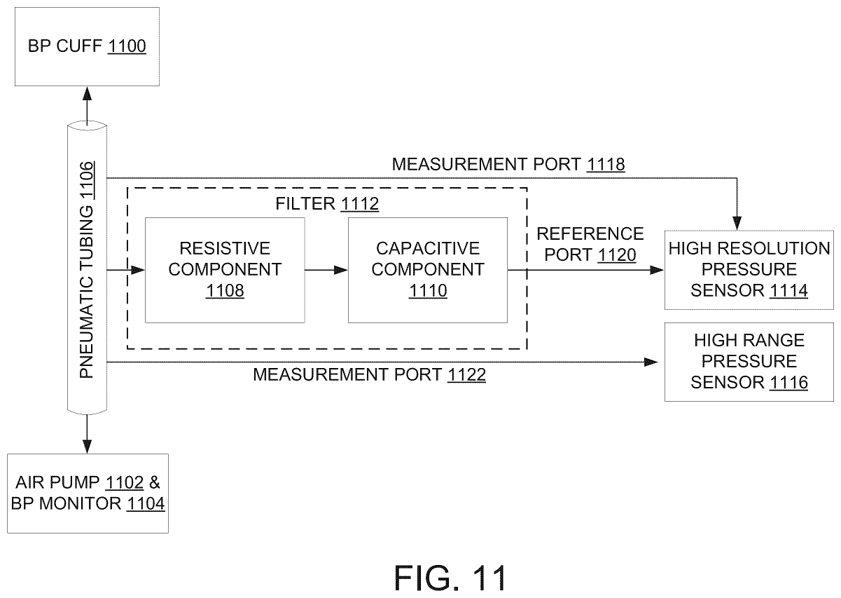

The BP cuff system can include a pneumatic assembly in combination with a BP monitor/cuff to capture the pulse pressure waveform in high resolution, enabling measurements of smaller pressure changes that some conventional systems. Particular implementations of a BP cuff system 20 that can be used to capture a pulse pressure waveform signal in enhanced resolution are further described below with reference to FIGS. 11-17, but it should be appreciated that the techniques described herein for performing central waveform reconstruction need not be limited to such systems, and they can be performed in conjunction with any BP cuff system or other suitable system that can capture pulse pressure waveform data of peripheral vascular site in sufficiently high resolution. This pulse pressure waveform signal can be stored in the digital domain as pulse pressure waveform data at BP cuff system 20 and/or waveform reconstruction device 10. In some implementations, the pulse pressure waveform data can be communicated from BP cuff system 20 to waveform reconstruction device 10 via a wired or a wireless communication link. For example, a radio frequency link such as a Bluetooth® link or a Wi-Fi® link can be used to communicate the data.

The waveform reconstruction device 10 is configured to process the pulse pressure waveform signal associated with the peripheral vascular site of subject 1 (“peripheral waveform” 11), and reconstruct an estimated pulse pressure waveform signal associated with a central vascular site of subject 1 (“central waveform” 12). For example, the pulse pressure waveform at the ascending aorta or left ventricle of subject 1 can be reconstructed given the measured pulse pressure waveform of the brachial artery of subject 1. FIG. 1B illustrates one particular embodiment in which pulse pressure waveform signal 13 measured for a brachial artery of a subject is directly measured using a BP cuff system 20, and reconstructed into an estimated pulse pressure waveform signal 14 for a LV of the subject using waveform reconstruction device 10.

Data corresponding to the peripheral waveform 11 and/or central waveform 12 can be stored at waveform reconstruction device 10. The waveform reconstruction device 10 can further process the data associated with the central waveform 12, e.g., by performing PWA, to assess the cardiac health of the subject 1 and/or predict whether or not the subject 1 potentially suffers from a cardiac condition. In some implementations, the waveform reconstruction device 10 can extract waveform parameters from peripheral waveform 11 prior to reconstruction of central waveform 12. These extracted waveform parameters can be used during reconstruction to enhance the accuracy of the reconstructed central waveform 12. In some implementations, the waveform reconstruction device 10 can store and/or retrieve physiological parameters (e.g., age, weight, gender) of the subject 1. These physiological parameters can be used during reconstruction (without or without the waveform parameters) to enhance the accuracy of the reconstructed central waveform 12. The particular methods that can be implemented by waveform reconstruction device 10 to reconstruct central waveform 12 and/or assess the cardiac condition of subject 1 based on the reconstructed central waveform 12 are further described below.

Also illustrated in the system of FIG. 1A is an imaging device 30 that is configured to capture one or more images of the central vascular site of subject 1. The imaging device 30 can be used, for example, in implementations where LV PV loops are generated for subject 1. In such implementations, imaging device 30 can use non-invasive imaging modalities such as ultrasound imaging or magnetic resonance imaging (MRI) to obtain images from which an LV volume curve is derived. For example, imaging device 20 can be used to perform echocardiography of subject 1 using ultrasound imaging, and the LV volume curve can be derived from the echocardiography images. In such implementations, the BP cuff system 20 can be used to non-invasively obtain a reconstructed LV pressure curve. Thereafter an LV PV loop can be derived from the LV volume and pressure curves, and the ventricular function and other heart health parameters can be assessed by examining the PV loop. The particular methods that can be implemented to derive an LV PV loop are further described below. In some implementations, the imaging data captured by imaging device 30 can be communicated from imaging device 30 to waveform reconstruction device 10 via a wired or a wireless communication link. In some implementations, the waveform reconstruction device 10 can derive the LV volume curve from the imaging data. The waveform reconstruction device 10 can also derive the LV PV loop. In some implementations, e.g., where PV loops of the LV are not generated, imaging device 30 can be omitted from the system.

In this particular example, waveform reconstruction device 10 is illustrated as a mobile device in communication with BP cuff system 20. The mobile device can be a smartphone, a tablet, laptop, a smartwatch, a head mounted display (HMD), or other suitable mobile device configured to generate the reconstructed central waveform 12. In a particular implementation, the mobile device can run an application for performing waveform reconstruction. The application can be configured to display the pulse pressure waveform data of the peripheral and/or central vascular sites. The application can also be configured to display a cardiac condition and/or expected diagnosis based on processing the central waveform 12. In other implementations, a desktop computer or other device can be implemented as waveform reconstruction device 10. In yet other implementations, waveform reconstruction device 10, BP cuff system 20, and/or optional imaging device 30 can be integrated as part of the same system. For example, the functions and/or hardware of waveform reconstruction device 10 can be incorporated into BP cuff system 20. In some implementations, waveform reconstruction device 10 can be a device specifically dedicated to performing central waveform reconstruction. For example, the device 10 can be designed with an integrated circuit that specifically performs the central waveform reconstruction functions described herein. In such implementations, signal processing could take place in the analog domain.

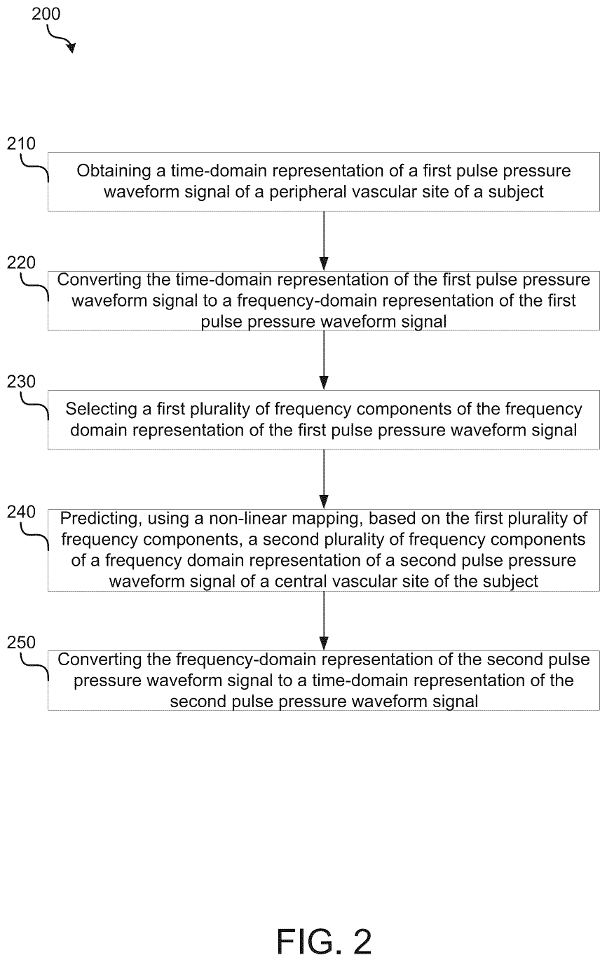

FIG. 2 is an operational flow diagram illustrating an example method 200 that can be implemented to reconstruct an estimated pulse pressure waveform associated with a central vascular site of subject, given a measured pulse pressure waveform associated with a peripheral vascular site of the subject, in accordance with some implementations of the disclosure. In some implementations, one or more of the operations of method 200 can be performed by cardiac waveform reconstruction device 10 and/or BP cuff system 20 in response to executing, using a processing device, instructions stored thereon. In some implementations, method 200 can be implemented as two independent methods by BP cuff system 20 and waveform reconstruction device 10. For example, in some implementations operation 210 can correspond to a method that is performed by a BP cuff system to capture a pulse pressure waveform signal of a peripheral vascular site, and operations 220-250 can correspond to a subsequent method of central waveform reconstruction that can be performed by a waveform reconstruction device 10 using the pulse pressure waveform signal of the peripheral vascular site as an input. In some implementations, waveform reconstruction device 10 can perform operations 220-250 in response to executing instructions corresponding to an application for performing central waveform reconstruction.

Operation 210 includes obtaining a time-domain representation of a first pulse pressure waveform signal of a peripheral vascular site of a subject. The peripheral vascular site can be a peripheral artery such as a brachial artery. The time-domain representation of the first pulse pressure waveform signal can be obtained via a non-invasive measurement using a BP cuff such as a BP cuff of a BP cuff system 20.

In some particular implementations, the time-domain representation of the first pulse pressure waveform signal can be obtained as follows. Using a BP cuff of a BP cuff system (e.g., BP cuff system 20), a blood pressure measurement can obtain one or more blood pressure values corresponding to a subject. The blood pressure measurement can be performed using a brachial cuff that is placed on the subject's left or right arm. The blood pressure measurement can provide subject specific blood pressure values such as a SBP and/or a diastolic blood pressure (DBP). For example if the subject has a normal blood pressure, the subject specific blood pressure values can range between 90/60 millimeters of mercury (mmHg) and 120/80 mmHg. These blood pressure values can be used as a baseline. Specifically, the blood pressure values can provide a guideline of how the force that is applied from the cuff to the artery (e.g., brachial artery) will change the measured signal. Based on the one or more blood pressure values corresponding to the subject, the BP cuff can be inflated to a subject specific pressure value. While the BP cuff is inflated to the subject specific pressure value, the BP cuff system can capture the pulse pressure waveform signal associated with the brachial artery.

An arm cuff system that measures a pulse waveform with high resolution can be used. Such a design can be configured to use a blood pressure monitor with tourniquet capabilities to inflate and hold a specific pressure in the cuff. During the hold pressure period, a high-resolution pressure sensor can used to measure the pulse pressure waveform with fast enough sampling to record heart sounds. In various implementations, the subject specific pressure value is selected to remove other sources of noise besides the sounds created by the opening and closing of the valve. In particular, the subject specific pressure value can be selected to reduce or remove any noise created by local blood flow vibrations. To this end, in some implementations the subject specific pressure value can be selected to be greater than the measured SBP. This pressure above SBP can be referred to as a sSBP. The sSBP can be set to some threshold value above the measured SBP that ensures the artery is fully constricted. In a particular embodiment, the sSBP can be set to about SBP+35 mmHg.

When an internal pressure is present in the artery and an external pressure is applied that is greater than this internal pressure, the artery can be fully closed, removing any noise created by local blood flow vibrations. As such, a fully obstructive/occlusive pressure (cuff pressure greater than blood pressure) can be used to generate a signal that is free from local flow vibrations. By virtue of setting the subject specific pressure value to remove local noise, a cleaner measurement of the opening and closing of a heart valve can be obtained. In alternative implementations, the subject specific pressure value can be selected to be below sSBP. For example, the pressure-sound measurement can be performed at physiological hold pressures such as, but not limited to, sub-DBP, DBP, mean arterial pressure (MAP), or SBP.

In some implementations, the pulse pressure waveform signal can be captured in the analog domain. In some implementations, the pulse pressure waveform signal can be captured as pulse pressure waveform data in the digital domain.

The peripheral pulse pressure waveform signal captured using the foregoing techniques can be a continuous signal corresponding to multiple cardiac cycles. In some implementations of operation 210, a pulse pressure waveform signal corresponding to a single cardiac cycle can be isolated from such a continuous signal. A cardiac cycle can refer to the sequence of events that occur from the start to the end of a heartbeat. The cardiac cycle is typically broken down into systolic and diastolic phases, which are the contraction and relaxation phases, respectively. These series of event generate specific pressure characteristics that can be used to segment and isolate individual cardiac cycles. The cardiac cycle typically starts in systole, which is marked by a steady pressure rise. The cardiac cycle then progresses to diastole, where the aortic valve closes and pressure steadily drops. Cardiac cycles can be isolated by identifying the start of the pressure rise.

Operation 220 includes converting the time-domain representation of the first pulse pressure waveform signal to a frequency-domain representation of the first pulse pressure waveform signal. Operation 230 includes selecting a first plurality of frequency components of the frequency domain representation of the first pulse pressure waveform signal. Various techniques can be utilized to perform the time-frequency transformation, including wavelet decomposition, empirical mode decomposition, Fourier transformation, and the like.

The time-frequency transformation to obtain the first plurality of frequency components can be applied as follows. A time-domain representation of the first pulse pressure waveform signal that corresponds to a cardiac cycle can be decomposed into a first number of frequency modes using a time-frequency analysis tool such as the FFT. The first n Fourier harmonic modes can be selected such that the error on self-reconstruction of the pressure waveform from its Fourier modes is below a predetermined threshold. For example, in some implementations, the number of Fourier harmonic modes can be selected to be 5 or more as below 5 modes the error can potentially significantly increase. In some implementations, a maximum number of Fourier harmonic modes can also be set considering the marginal improvements provided by increasing the number of modes versus the use of computational resources.

Operation 240 includes predicting, using a non-linear mapping, based on the first plurality of frequency components, a second plurality of frequency components of a frequency domain representation of a second pulse pressure waveform signal of a central vascular site of the subject. The central vascular site can correspond to a LV or ascending aorta of the subject. Any suitable non-linear mapping that can account for the non-linear interaction of pressure waves propagating in the cardiovascular system can potentially be applied to obtain the second plurality of frequency components to construct the central waveform. For example, in some implementations, a machine learning model can be trained to predict the second plurality of frequency components associated with a frequency-domain representation of a pulse pressure waveform signal of a central vascular site, given the first plurality of frequency components of a frequency-domain representation of a pulse pressure waveform of a peripheral vascular site as an input. Some example machine learning techniques that could potentially be applied include regression-based techniques such as support vector regression, least absolute shrinkage, and selection operator. Other example machine learning techniques that could be applied include random forests or a gradient boosted decision tree. In some implementations, deep learning techniques such as neural networks, including convolutional neural networks, could be applied to generate the prediction.

In some implementations, a dataset including previous measurements of pulse pressure measurements obtained for both peripheral and central vascular sites of patients (e.g. using a BP cuff and invasive catheterization) could be used to train a model to make the foregoing prediction. For example, a model could be trained with the Fourier modes of known brachial waveforms as input and the Fourier modes of known left ventricular or ascending aorta waveforms as output.

It should be noted that the number of the second plurality of frequency components that is predicted for the frequency-domain representation of the second pulse pressure waveform signal can be the same or different from the number of the first plurality of frequency components. For example, “n” modes can be used as an input to generate “m” mode predictions, where n and m can be the same integer or different integers. Moreover, depending on the implementation, all of the “n” modes or only some of the “n” modes can be used to predict each “m” mode. In some implementations, the interaction of the predicted modes could also be considered when developing the non-linear mapping. As such, predicting the second plurality of frequency components can comprise individually or cumulatively predicting the second plurality of frequency components using some or all of the first plurality of frequency components as an input.

Operation 250 includes converting the frequency-domain representation of the second pulse pressure waveform signal to a time-domain representation of the second pulse pressure waveform signal. For example, in implementations where the FFT was applied to generate the time-frequency domain transformation, the predicted “m” modes corresponding to a respective cardiac cycle can be inverse FFTed to generate the predicted central waveform measurement.

Although described in the context of being applied to a single waveform signal corresponding to a single cardiac cycle, method 200 could be applied to a single waveform signal as well as a set of waveforms from the same subject. As such, application of this method to a sequential set of waveforms could enable generation of a continuous output pressure signal such as those used in hospital settings or wearable electronics.

To illustrate one example implementation of the foregoing method, it is instructive to consider a method for transferring a pressure wave a from a peripheral site to a left ventricular pressure waveform by applying a regressor on the wave harmonic content in the frequency space. To implement this particular method, the wave harmonic content for the LV pulse waveform can be generated given the wave components of the pressure-time signal from a peripheral measurement, e.g., such as from a brachial artery measurement. These harmonics can be converted to the time domain to yield the pressure-time signal inside the left ventricle. For example, a single cardiac cycle from a brachial measurement can be decomposed into n frequency modes using a FFT or other time-frequency analysis tool. The n modes can be used as an input to a regressor to generate m mode predictions for the left ventricular waveform. In some implementations, the number of left ventricular modes m can be selected to minimize the error on self-reconstruction. In some implementations, m can be set to be 10 or greater. For example, it was observed by the inventors that, in some implementations, the left ventricular waveform below 10 modes does not allow to capture the first derivative of the waveforms. The predicted m modes, with the length of the respective cardiac cycle, can be inverse FFTed to generate the predicted LV waveform measurement.

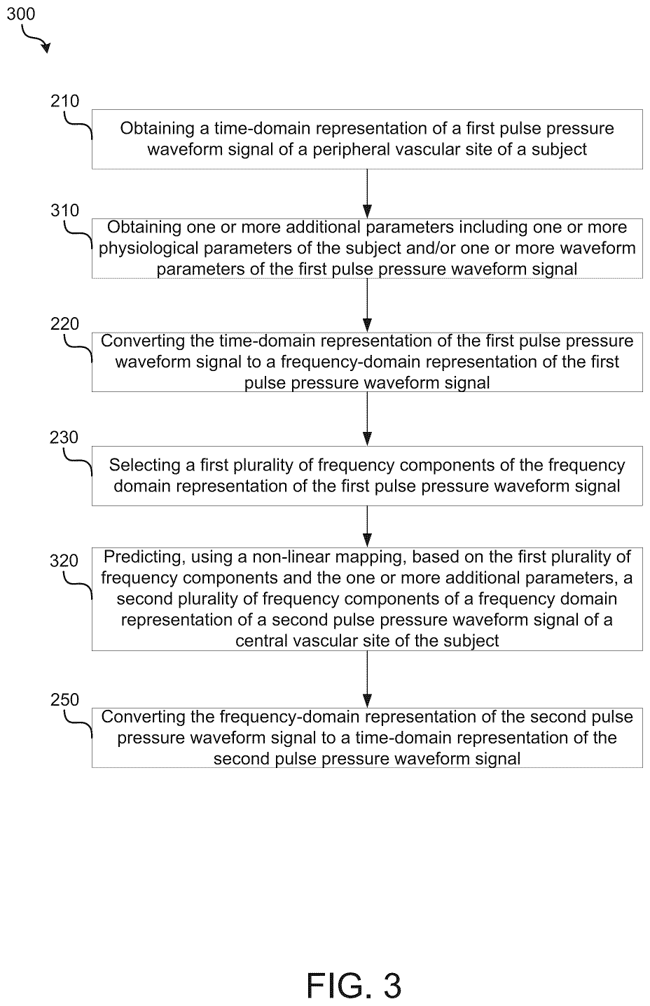

In some implementations of the disclosure, central waveform reconstruction accuracy can be improved by additionally providing the non-linear mapping with physiological information, herein referred to as physiological boosting. To this end, FIG. 3 is an operational flow diagram illustrating an example method 300 that leverages physiological boosting to reconstruct an estimated pulse pressure waveform associated with a central vascular site of subject, in accordance with some implementations of the disclosure. As depicted, operations 210, 220, 230, and 250 can be implemented in a similar manner as described above with reference to method 200. In addition to these operations, method 300 includes operations 310 and 320, which can be performed by cardiac waveform reconstruction device 10 or some other device. For example, operations 310 and 320 can be performed in response to executing, using a processing device, instructions stored thereon.

Operation 310 includes obtaining one or more additional parameters including one or more physiological parameters of the subject and/or one or more waveform parameters of the first pulse pressure waveform signal. Operation 320 includes predicting, using the non-linear mapping, based on the first plurality of frequency components and the one or more additional parameters, a second plurality of frequency components of a frequency domain representation of a second pulse pressure waveform signal of a central vascular site of the subject.

In this example, the one or physiological parameters of the subject can include an age, weight, height, or gender, or other physiological parameter of the subject that can be indicative of content of the waveform of the central vascular site of the subject. The one or more waveform parameters of the first pulse pressure waveform signal can be directly extracted from the time-domain representation of the first pulse pressure waveform signal. Such parameters can include a maximal or minimal rate of change over time, a pulse wave velocity, an area under the full curve or a segment of the curve, a time interval between two specific cardiac cycle events, magnitudes of specific points on waveform, measures related to wave reflection such as augmentation index, etc.

While the harmonic content in the frequency space can capture wave dynamics throughout the cardiovascular system, there are other cause-effect relationships between different components of the waveform from the central vascular site to the peripheral vascular site. As such, a trained model or other non-linear mapping whose inputs include a combination of the harmonic content and specific physiologically relevant pulse waveform features and/or physiological parameters of the subject could generate improved predictions of the harmonic content of the central vascular site. Such implementations could generate particularly notable improvements in predictions in which the two pressure sites are physically separated during the diastolic part of the cardiac cycle. In some implementations, physiological boosting can enable a trained model to extrapolate additional information to generate a more comprehensive prediction.

In implementations where physiological boosting is applied, a dataset including previous measurements of pulse pressure measurements obtained for both peripheral and central vascular sites of patients (e.g. using a BP cuff and invasive catheterization), along with previous measurements of physiological parameters of the patients and/or pulse waveform features, could be used to train a model to make the foregoing prediction. For example, a model could be trained with an input including the Fourier modes of known brachial waveforms, and the physiological parameters and/or pulse waveform features associated with the known brachial waveforms. The output for training could be the Fourier modes of known left ventricular or ascending aorta waveforms.

In some implementations of the disclosure, PWA can be applied to the reconstructed central waveform to evaluate a cardiac condition of the subject. PWA is a methodology used to extract quantitative parameters from a continuous pressure time signal. Quantification of specific sections of the cardiac pulse waveform can allow interrogation of different aspects of the cardiovascular system. In cardiology, PWA has been used by clinicians to non-invasively interrogate properties of the cardiac system such as arterial stiffness and contractility.

Superposition of forward and reflected waves causes the waveform shape to be dependent on the measurement location. Considering cardiologists are concerned with the stress exerted on and in the heart, pressures closer to the source may generate the more accurate measurements. To this end, PWA can be applied on the central waveforms generated from the techniques described herein (e.g., method 200 or method 300) to extract pulse waveform features that are analyzed.

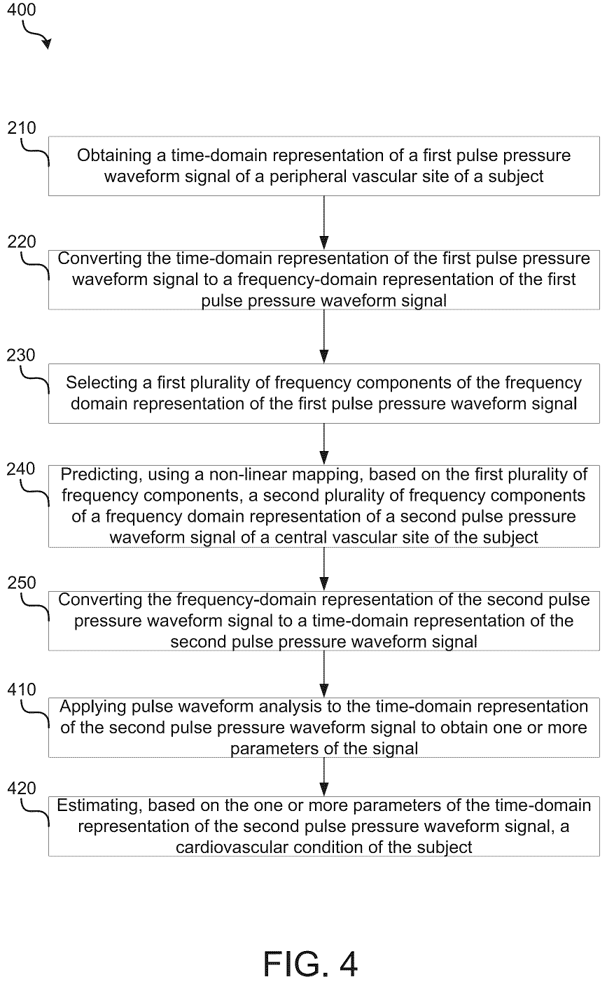

To this end, FIG. 4 is an operational flow diagram illustrating an example method 400 that performs PWA of an estimated pulse pressure waveform associated with a central vascular site of subject, to measure a patient cardiovascular condition, in accordance with some implementations of the disclosure. As depicted, operations 210-250 can be implemented in a similar manner as described above with reference to method 200. Alternatively, the operations of method 300 can be performed. In addition to these operations, method 400 includes operations 410 and 420, which can be performed by cardiac waveform reconstruction device 10 or some other device. For example, operations 410 and 420 can be performed in response to executing, using a processing device, instructions stored thereon.

Operation 410 includes applying pulse waveform analysis to the time-domain representation of the second pulse pressure waveform signal to obtain one or more parameters of the signal. Operation 420 includes estimating, based on the one or more parameters of the time-domain representation of the second pulse pressure waveform signal, a cardiovascular condition of the subject. Depending on the location of waveform reconstruction, different characteristics of the cardiovascular system can be inferred from the PWA. As a first example, PWA on the reconstructed central aortic pressure can estimate the arterial stiffness or the subendocardial viability ratio. As another example, PWA on the reconstructed left ventricular pressure can estimate the ventricular contractility as well as the left ventricular end diastolic pressure. Different PWA methodologies including, but not limited to, wave separation analysis, wave power analysis or wave intensity analysis, can be used to extract a multitude of parameters from the reconstructed waveforms. The accuracy of the extracted parameters from all different types of PWA can be directly dependent on the accuracy of the waveform and its morphology. As such, by virtue of the techniques described herein for improving the accuracy of the reconstruction methodology, the quality of the non-invasive diagnosis can be improved.

In some implementations of the disclosure, a PV loop of the LV of a patient can be generated non-invasively. LV PV loops describe the relationship between pressure and volume in the LV during a cardiac cycle. The PV loop provides a method for understanding cardiac mechanics and ventricular function and allows for measurement of work done by the heart. Although, theoretically, both the LV pressure and volume waveforms can be independently analyzed to extract parameters that characterize ventricular function, given the load dependency of these parameters, the joint analysis of the pressure and volume in the PV loop yields more accurate measures of cardiac functions.

Constructing a LV PV loop requires the measurement of both LV pressure and volume. One measurement approach utilizes PV catheters that simultaneously measure pressure and volume by using a pressure sensor and the conductance technique, respectively. PV catheters are challenging to use in clinical practice due to their invasiveness, the need for multiple calibration procedures, the measurement sensitivity to catheter placement in the heart chamber, and the artifacts present in the measurement. Another approach is to perform separate pressure and volumetric measurements via catheterization and imaging, respectively. While imaging can be performed with echocardiography or Magnetic Resonance Imaging, both of which are non-invasive, measurement of LV pressure via catheterization is an invasive procedure. The invasiveness of the pressure measurement severely limits practical applications.

Although one method proposed to use an empirical, normalized population-averaged reference LV pressure curve that is scaled for amplitude and durations the empirical waveform did not reflect patient-specific pressure waveform shapes, which are an important part of cardiac mechanics. Another approach proposed using a time-varying elastance model for generating non-invasive individualized PV loops. While the elastance model attempted to generate a patient specific pressure curve shape, the general elastance curve shape was obtained from optimized population averages and contained curve features that must be guessed or manually constrained. Both methods relied on restrictive assumptions and population-averaged curve morphologies which inevitably decrease the fidelity of the reconstructed waveform. Furthermore, both methods used brachial systolic blood pressure for LV peak value scaling and did not account for wave dynamics that are known to affect pressure magnitudes across the arterial system. As such, in the context of non-invasive LV PV loops, there is a clear need for a non-invasive method to reconstruct patient-specific LV pressure waveform magnitude and shape to increase the faithfulness of the reconstructed PV loop.

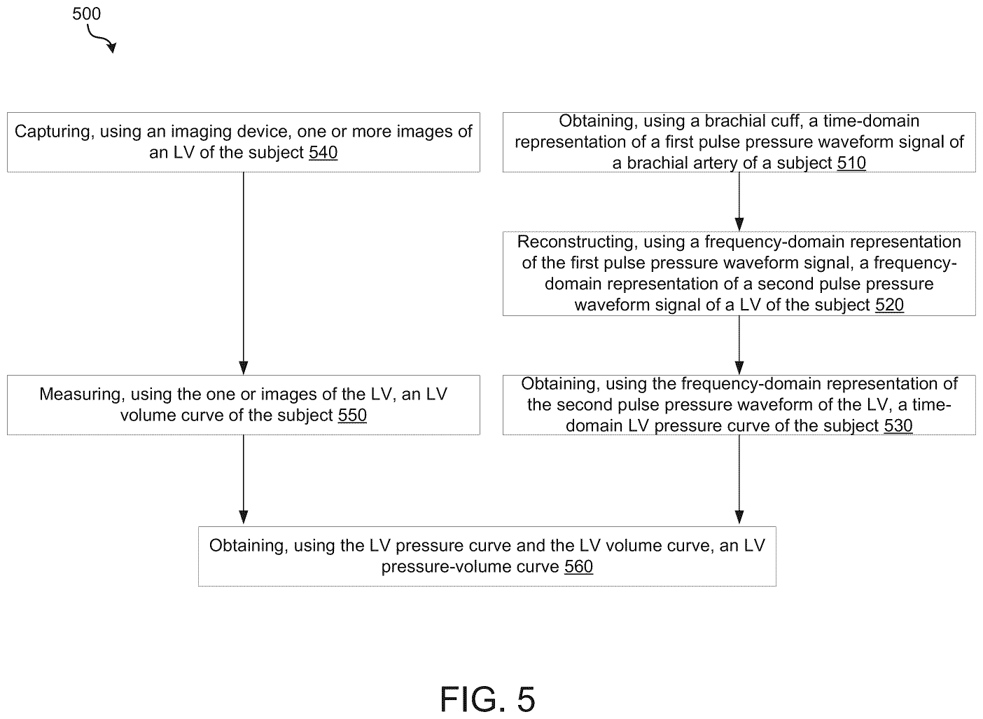

To this end, some implementations of the disclosure are directed to systems and methods for non-invasively reconstructing an individualized LV PV loop using non-invasive imaging modalities, a brachial cuff, and an optional ECG. FIG. 5 is an operational flow diagram illustrating one example method 500 for the reconstruction of a non-invasive LV PV loop of a subject, in accordance with some implementations of the disclosure. In some implementations, the operations of method 500 can be performed using waveform reconstruction device 10, BP cuff system 20, and imaging device 30. In some implementations, an optional ECG could also be used to measure the electrical activity of the subject's heart, which can be used for synchronizing measurements obtained using the brachial cuff and the imaging device.

To non-invasively obtain an LV pressure curve of the subject, operations 510-530 can be performed using a brachial cuff system. These operations can leverage the techniques previously described herein to generate high-fidelity patient-specific LV pressure waveforms using a non-linear mapping technique for patient-specific reconstruction of LV pressure waveform magnitude and shape from a non-invasive brachial measurement. Operation 510 includes obtaining, using a brachial cuff, a time-domain representation of a first pulse pressure waveform signal of a brachial artery of a subject. Operation 520 includes reconstructing, using a frequency-domain representation of the first pulse pressure waveform signal, a frequency-domain representation of a second pulse pressure waveform signal of a LV of the subject.

In some implementations, operation 510 can be implemented in a similar manner as described above with reference to operation 210 of method 200 when utilizing a BP cuff system that performs pressure measurements of the brachial artery. For example, a brachial cuff can perform an oscillometric blood pressure measurement followed by tourniquet mode for pulse waveform acquisition with at least one pressure hold in suprasystolic mode. Brachial waveform(s) from the suprasystolic mode can be statically or dynamically calibrated using the oscillometric blood pressure values to obtain a calibrated waveform in the time domain.

Operation 520 can be implemented in a similar manner as described above with reference to operations 220-240 of method 200 or operations 310, 220-230, and 320 of method 300. For example, the calibrated waveform in the time domain will be transformed to the frequency domain by utilizing a frequency decomposition methodology, such as the Fourier transform, wavelet transform, or empirical mode decomposition. A select number of modes can serve as input to a machine learning model for the prediction of m number of pressure curve modes at the LV site, where the number of modes n and m are independent. The reconstruction of the LV pressure waveform using the machine learning model could also be augmented with pulse waveform analysis parameters as described above.

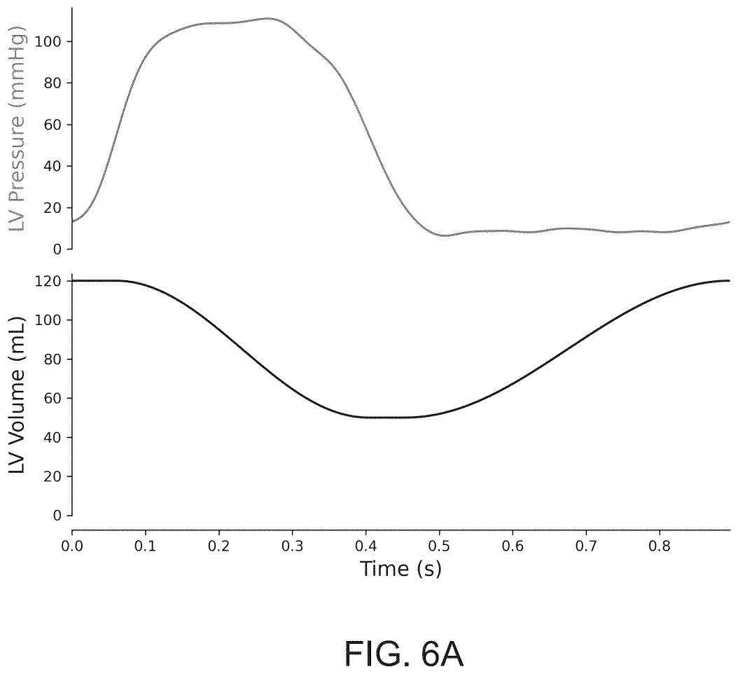

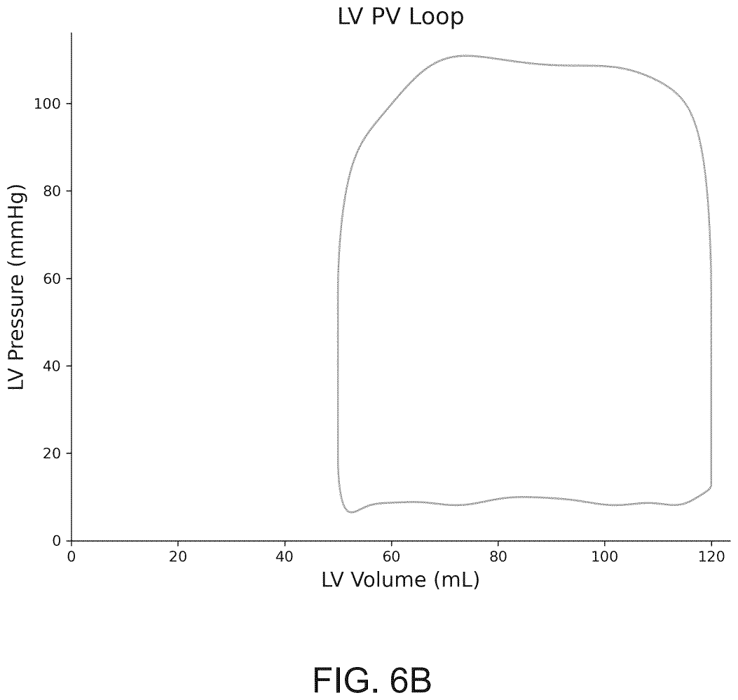

Operation 530 includes obtaining, using the frequency-domain representation of the second pulse pressure waveform of the LV, a time-domain LV pressure curve of the subject. Operation 530 can be implemented in a similar manner as described above with reference to operation 250 of method 200. For example, the LV pressure curve can be generated by transforming the predicted modes from the frequency to the time domain, e.g., using the inverse Fourier transform adjusted for the length of the respective cardiac cycle. In some implementations a single LV pressure curve or multiple LV pressure curves of the subject could be generated. FIG. 6A depicts one example of an LV pressure curve that can be generated using the techniques described with reference to FIG. 5.

To non-invasively obtain an LV volume curve of the subject, operations 540-550 can be performed. Operation 540 includes capturing, using an imaging device, one or more images of an LV of the subject. For example, an imaging device 30 as described above can be utilized. Operation 550 includes measuring, using the one or images of the LV, an LV volume curve of the subject. Imaging modalities, such as echocardiography or MRI, can be used by the imaging device for non-invasive measurement of the LV volume curve. For example, in some implementations of operation 540, an echocardiography procedure can capture the LV structure over one or multiple cardiac cycles. For volumetric measurements, both 2D and 3D echocardiography methods can be used with the non-contrast or contrast-enhanced modalities. LV borders or volume can be detected from the captured images, and an LV volume curve can be obtained. FIG. 6A depicts one example of an LV volume curve that can be captured using the techniques described with reference to FIG. 5.

Operation 560 includes, obtaining, using the LV pressure curve and the LV volume curve, an LV pressure-volume curve corresponding to the subject. FIG. 6B depicts one example of an LV pressure-volume curve that can be generated from the LV pressure curve and LV volume curve of FIG. 6A, using the techniques described with reference to FIG. 5. In some implementations, operation 560 includes time synching the LV volume and pressure curves followed by segmentation of the LV pressure and volume curves. In other implementations, time synching may not be required.

Depending on the implementation, the imaging device image captures (e.g., echocardiography) and brachial cuff measurements can be performed either simultaneously, or at different instances and posteriorly time synched. Measurements at different instances can be time synched using cardinal points on the pressure and volume waveforms, such as the opening and closure of the heart valves. Measurements at different instances can also be time synched by using information from a third independent signal, such as an optional ECG. In this implementation, the ECG signal can be measured with both echocardiography and brachial cuff. Upon completion of time-synching, if necessary, LV pressure and volume curves can be segmented starting from a common event to isolate one or more cardiac cycles.

The time-synched pressure and volume curves can be plotted simultaneously on a pressure-volume curve. In some implementations, for a given subject one or more cardiac cycle PV loops can be obtained, plotted, and/or averaged for analysis. The reconstructed, non-invasive PV loop can be analyzed by a physician or algorithmically (e.g., using device 10) to diagnose cardiovascular conditions. In some implementations, multiple PV loops from different time instances could be used to diagnose and monitor chronic cardiovascular conditions.

### Experimental Results

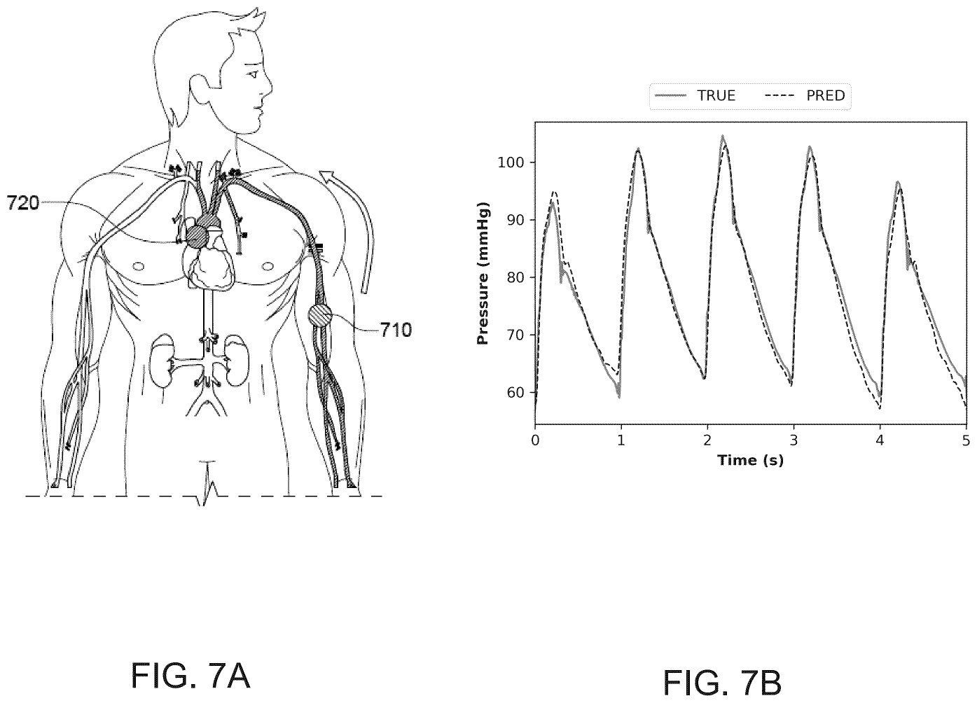

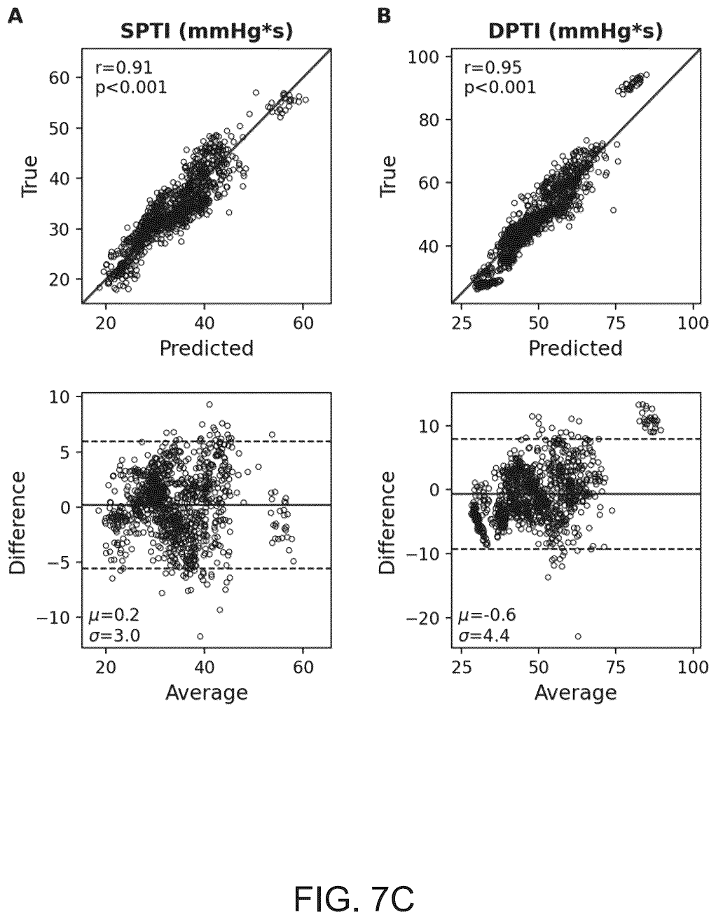

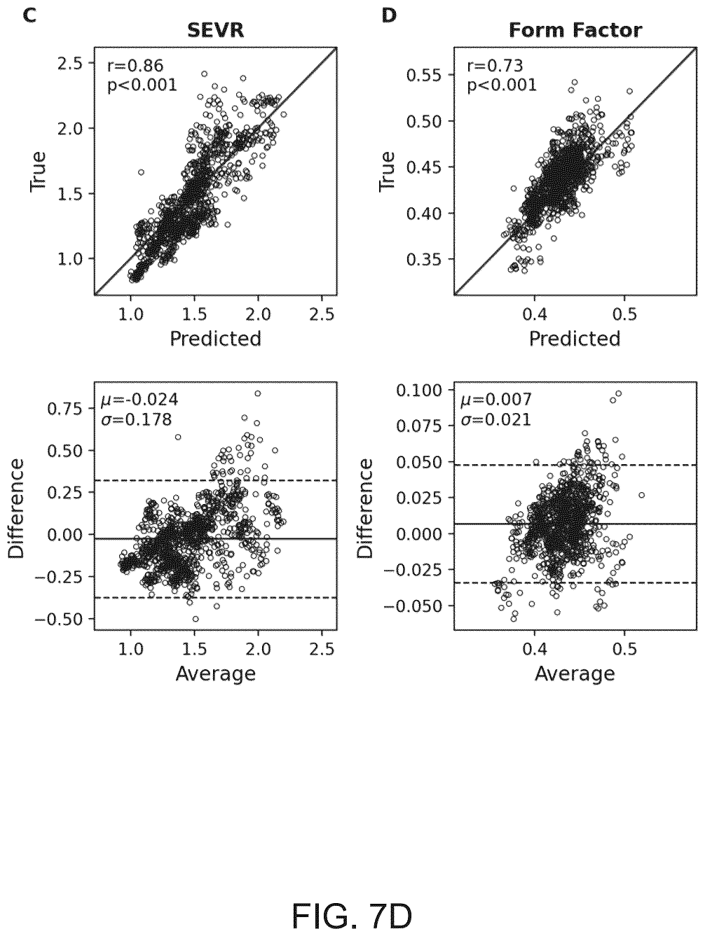

FIGS. 7B-7D illustrate experimental results associated with central aortic (ascending aorta) pulse pressure waveforms reconstructed using measured brachial pulse pressure waveforms, in accordance with some implementations of the disclosure. FIG. 7A is a diagram illustrating a brachial site 710 of a subject where a pulse pressure waveform measurement can be captured using a BP cuff system, and the central aortic site 720 for which the pulse pressure waveform can be reconstructed for the subject, in accordance with some implementations of the disclosure.

FIG. 7B shows the predicted pulse pressure waveform at the ascending aorta reconstructed using a measured brachial pulse pressure waveform, and the actual pulse pressure waveform measurement captured using an invasive catheter placed in the ascending aorta. FIGS. 7C-7D include plots illustrating the accuracy of pulse waveform parameters measured using PWA on the reconstructed waveform when compared to those measured on the actual waveform measured with a catheter in the ascending aorta. PWA was applied to all waveform pairs (predicted aortic waveform and measured aortic waveform) in the testing dataset to extract waveform parameters. These waveform parameters were then plotted using a true (aka measured one) versus predicted using the techniques described herein (top plot) and a bland-altman analysis (bottom plot). The plots represent the systolic pressure time integral (SPTI), the diastolic pressure time integral (DPTI), the subendocardial viability ratio (SEVR), and the form factor, respectively. As the results show, the parameters measured on the reconstructed waveform accurately match those measured on the actual measured waveform, indicating that the reconstruction techniques described herein can provide reliable and accurate waveforms. As doctors use these parameters in the clinic, being able to capture these parameters non-invasively could have significant clinical advantages.

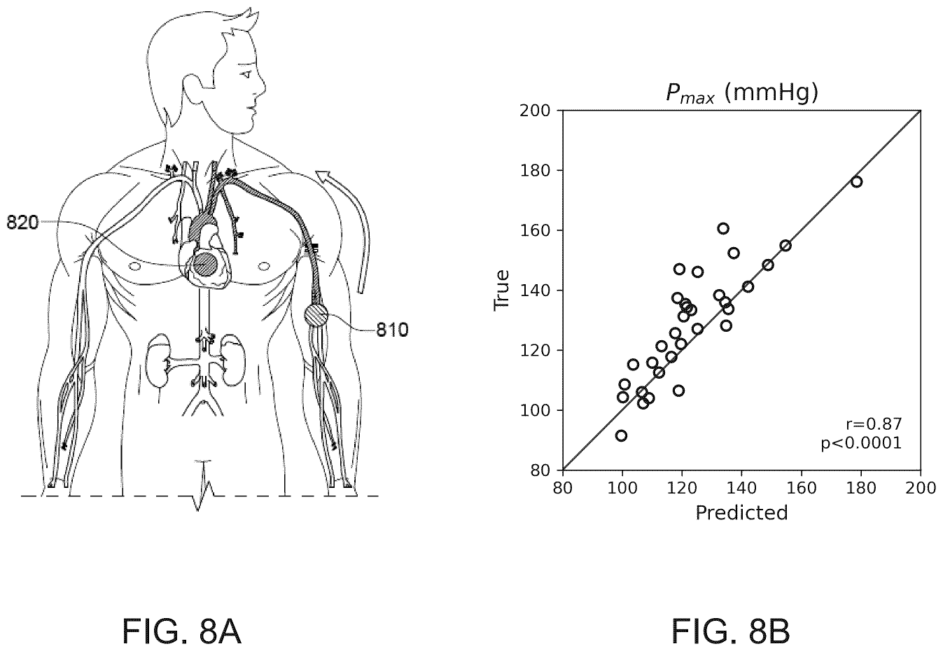

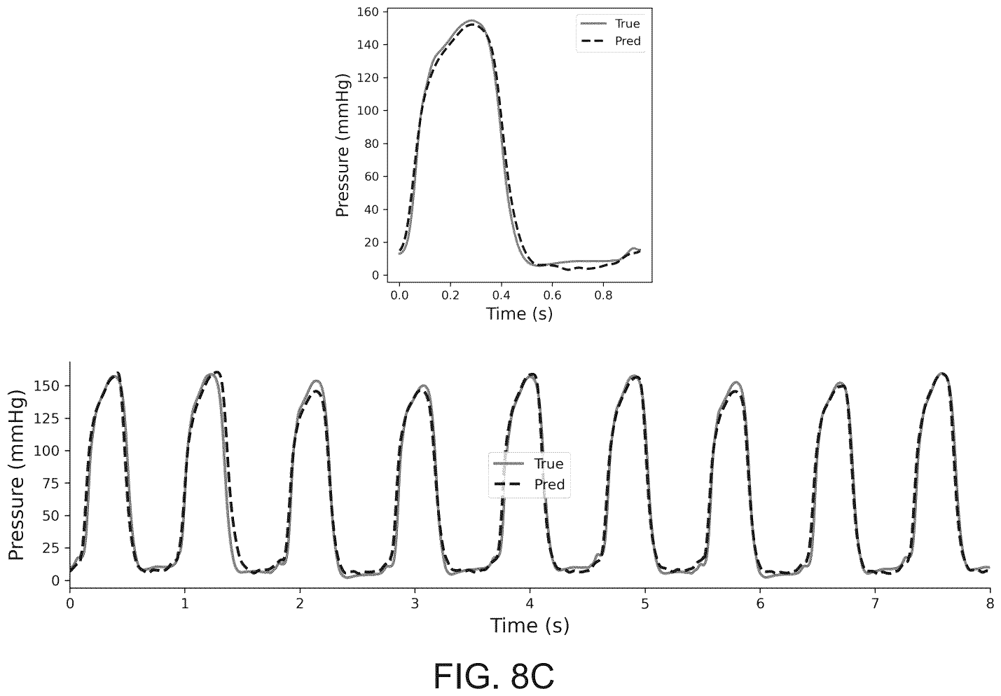

FIGS. 8B-8C illustrate experimental results associated with LV pulse pressure waveforms reconstructed using measured brachial pulse pressure waveforms, in accordance with some implementations of the disclosure. FIG. 8A is a diagram illustrating a brachial site 810 of a subject where a pulse pressure waveform measurement can be captured using a BP cuff system, and the LV site 820 for which the pulse pressure waveform can be reconstructed for the subject, in accordance with some implementations of the disclosure.

FIG. 8B includes a comparison plot showing the true (measured) values versus predicted values for left ventricular maximum pressure. True waveforms were measured on patients in a clinical environment. A catheter was inserted and positioned inside the left ventricle, providing a direct reading of the pressure inside the left ventricle, which is considered the true measurement. Predicted values for the maximum pressure in the left ventricle were obtained using techniques described in the disclosure. FIG. 8C includes a first plot showing an example of a reconstructed and measured left ventricular pressure waveform for a single cardiac cycle (top), and a second plot showing an example of reconstructed and measured left ventricular pressure waveforms for multiple sequential cardiac cycles representing a continuous measurement modality (bottom). As the foregoing results illustrate, it is possible to non-invasively reconstruct the left ventricular pressure waveform using a brachial cuff system. By contrast, current techniques for measuring LV pressure rely on placement a catheter inside the heart of the patient, which is invasive.

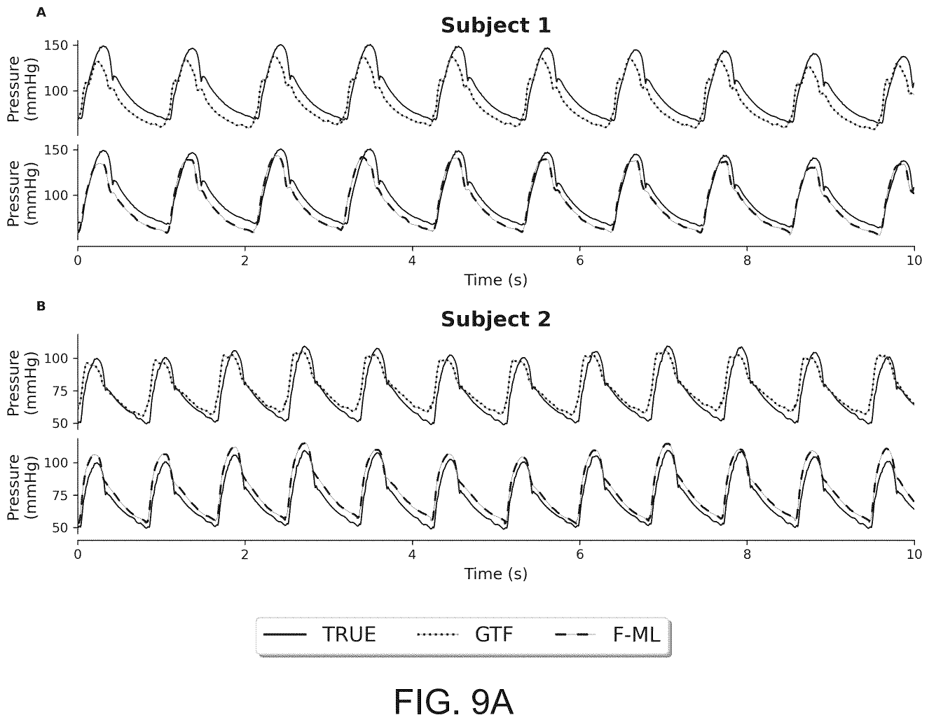

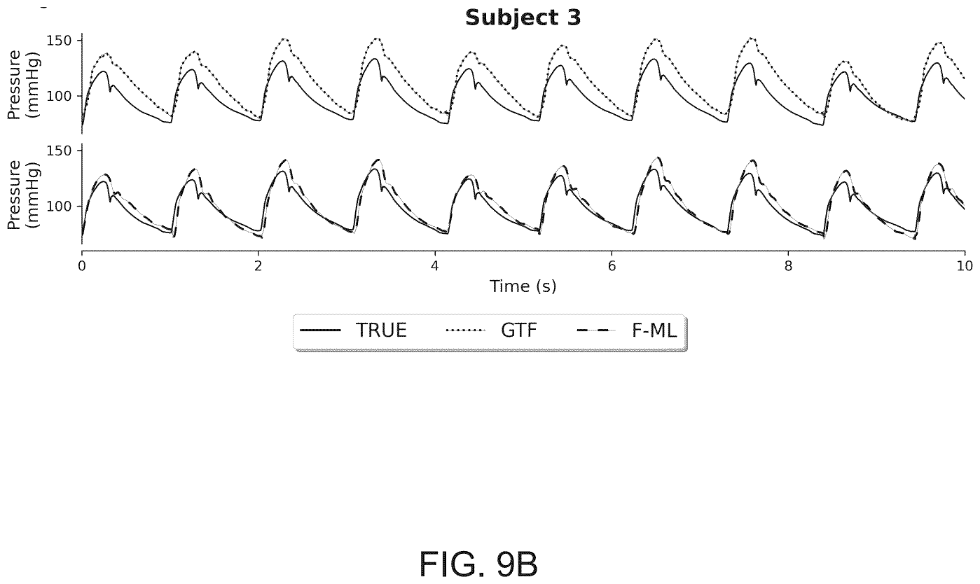

FIGS. 9A-9B are plots showing a ten second segment of a continuous pulse waveform measurements obtained for three subjects using a catheter (TRUE), versus the reconstructed pulse waveforms using the GTF and using Fourier-based machine learning methods in accordance with the disclosure. The plots were generated using a dataset containing brachial cuff and invasive central catheter waveforms. This dataset included simultaneous brachial and central (ascending aorta) pulse waveform measurements from 115 subjects. The dataset was split into training and testing data (70% and 30% respectively), of which the testing was data used once only for validation. FIGS. 9A-9B show a sample of this dataset, including the continuous transformation of the peripheral measurement to central waveform and the comparison with the conventional GTF. Overall, the results show that the proposed method generates waveform predictions for central pressure with superior accuracy compared with current standard methods.

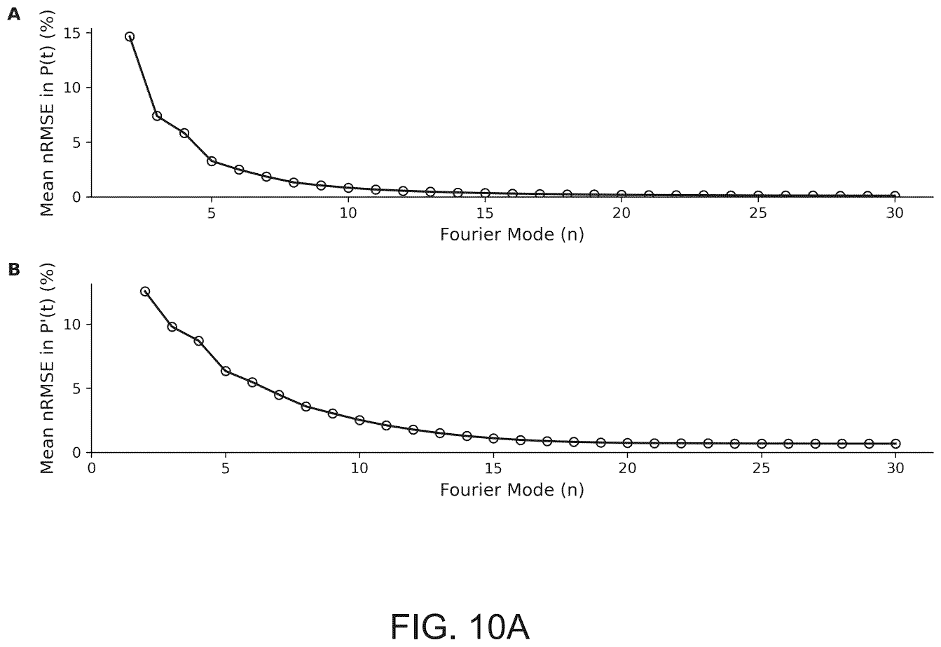

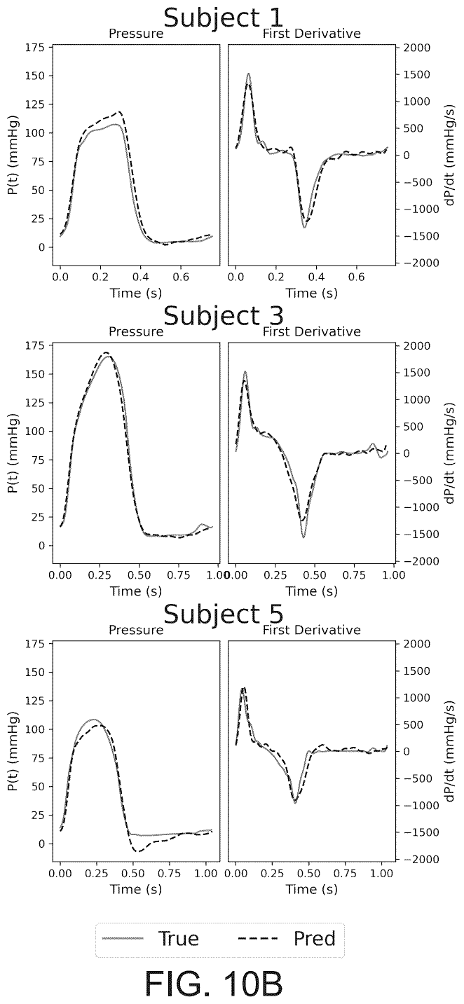

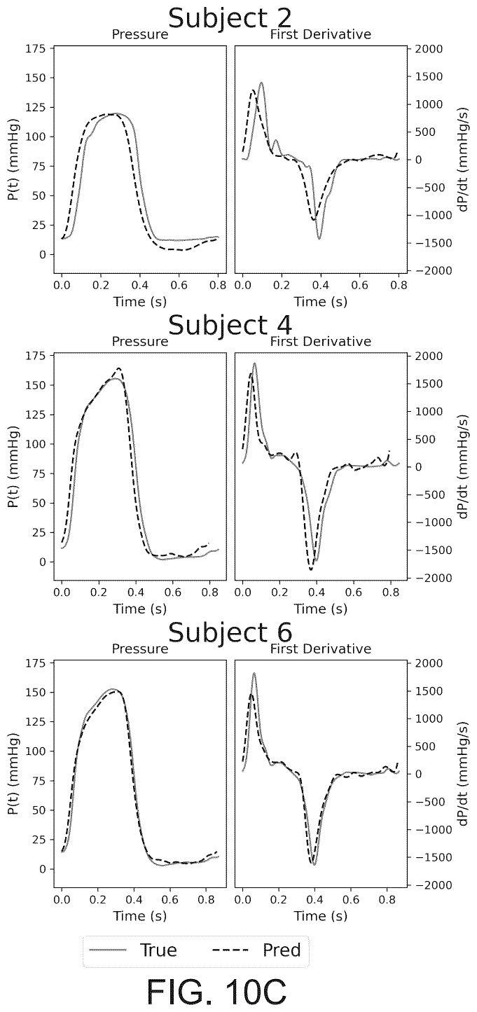

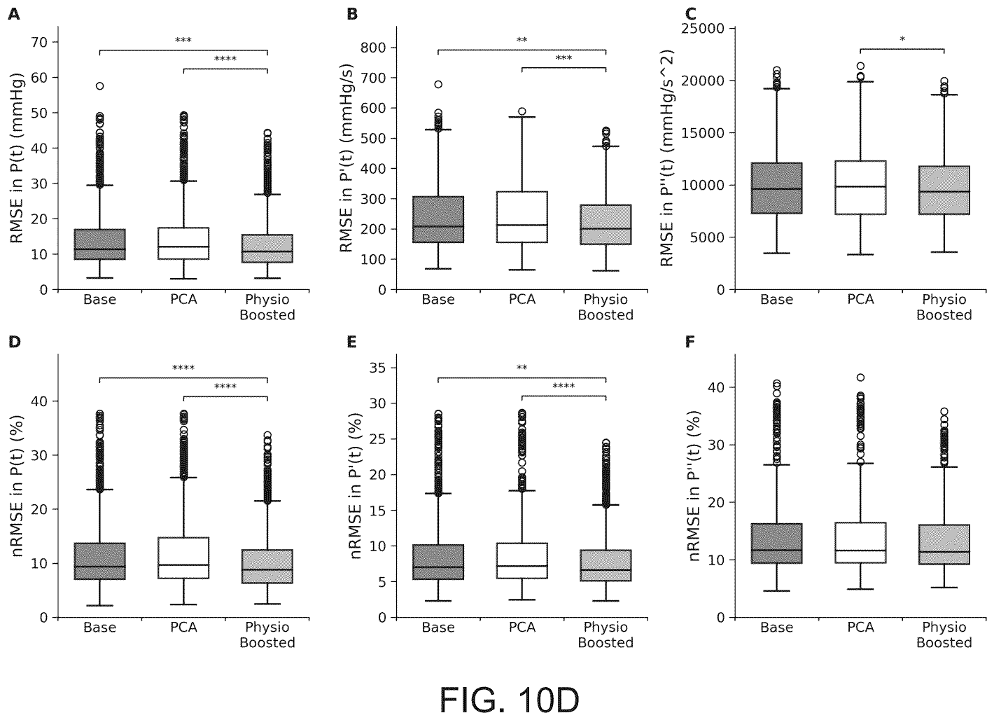

FIG. 10A are plots showing the nRMSE between the measured left ventricular waveform and the self-reconstructed waveform based on a different number of Fourier modes, in accordance with some implementations of the disclosure. As depicted, analysis was performed both for the pressure-time signal (top plot) and the first derivative of the pressure time signal (bottom plot). In this particular implementation, single cardiac cycles from brachial peripheral measurements were decomposed into frequency modes using the FFT. The frequency modes were used as an input to a machine learning model to generate m mode predictions for the left ventricular waveform. As depicted, the error on self-reconstruction appeared to plateau after about 10 modes, where below 10 modes the error increased significantly with the use of fewer modes. This was valid for both the pressure time signal and the first derivative.

A method for reconstruction of the left ventricular pulse waveform from a peripheral measurement at the brachial artery was implemented on real human data consisting of a dataset containing brachial cuff waveforms and invasive waveforms from a catheter in the left ventricle. This dataset included simultaneous brachial and left ventricular pulse waveform measurements from 104 subjects. The dataset was split into training and testing data (70% and 30%, respectively), of which the testing was data used once only for validation. FIGS. 10B-10D illustrate the results of the implementation of the methods described herein on this dataset.

FIGS. 10B-10C include six examples showing the comparison between the measured and reconstructed left ventricular pulse waveforms (left panel of each figure) and the first derivative (right panel of each figure) utilizing the Fourier-based machine learning approach described herein. FIGS. 10B-10C demonstrate the capability of the Fourier-based machine learning model to transform the peripheral measurement from the brachial artery waveform to left ventricular waveform. The comparison of the reconstructed pressure-time signal and its first derivative with the measured signals shows great agreement indicating that the method can capture the left ventricular dynamic with strong accuracy.

FIG. 10D illustrates a comparison of the results from three different F-ML structures inclusive of a Base F-ML model, a F-ML model with principal component analysis to reduce dimensionality (PCA), and a F-ML model with physiological boosting (Physio Boosted). The top row of figures shows the reconstruction error measured with the Root-Mean-Squared-Error (RMSE) on the pressure time signal (a), its first derivative (b), and its second derivative (c). The bottom row of figures shows the reconstruction error measured with the nRMSE on the pressure time signal (d), its first derivative (e), and its second derivative (f). Normalization of RMSE was performed with the peak-to-peak signal amplitude. Significance levels: *p<0.05, **p<0.01, ***p<0.001. As depicted, the addition of physiological parameters to the F-ML model produces improvements that are statistically significant both in the pressure-time signal and its first derivative. The foregoing results illustrate that the methods of the disclosure can be successfully applied to the reconstruction of the left ventricular waveform and show that physiological boosting can enhance model prediction accuracy,

### Bp Cuff System

Embodiments of the systems and methods described herein can utilize a BP cuff system including a modified BP cuff to perform non-invasive yet accurate cardiac measurements. The BP cuff system can be leveraged to perform pulse pressure waveform measurements as described herein. The BP cuff system can also perform left ventricular end-diastolic pressure (“LVEDP”) measurements, pressure-volume (“PV”) loop measurements, and other important cardiac measurements. The BP cuff system can be used in conjunction with methods for performing central waveform reconstruction as described herein.

### Non-Invasive Pulse Pressure Waveform Measurement

The modified BP cuff system may be used to measure a pulse pressure waveform. In some implementations, the modified BP cuff system may include a dynamic pressure sensor instead of and/or in addition to a static pressure sensor. A high resolution pressure sensor may be included for high sensitivity signal acquisition at a specified pressure level. The high resolution pressure sensor may comprise a differential pressure sensor having a measurement port and a reference port, wherein the pressure sensor measures the difference between its measurement port and reference port. A high range absolute pressure sensor may be used to calibrate the signal. An air valve or filter may be included to maintain a specific pressure level at the reference port. Maintaining the pressure level may allow the high resolution pressure sensor to operate within its normal range. During measurement the pressure sensors may simultaneously acquire signals.

The high range pressure sensor may measure with respect to atmospheric pressure while the high resolution pressure sensor may measure with respect to a variable reference pressure. The pressure sensors may be connected in parallel to the BP cuff system. In some implementations, the high range and high resolution pressure sensors may have an operating range on the order of magnitude of the measurement and signal, respectively.

No control system may be employed in the implementations described herein. Although such control systems could be used to dynamically control an air valve to open and close the valve at appropriate pressures to ensure the signal is captured correctly, in such a system, pressure fluctuations may cause sensor saturation resulting in a critical fault, signal drift, and loss of valuable information. For these reasons, an air valve system may present drawbacks that are not present in the implementations described herein.

In some implementations, a passive and self-adjusting non-invasive pulse pressure waveform measurement system may include high resolution pressure sensors in an inflatable pressurized air chamber having resistive and capacitive components. Hydraulic filtering may be implemented through a geometrical condition to passively generate a signal that only transits desired frequencies. In some embodiments an air valve may be replaced with a hydrodynamic filter. A hydrodynamic filter may comprise a fixed or adjustable orifice, an in-line filter, and/or tubing with an internal diameter (“ID”) significantly smaller than the ID of the rest of tubing included in a modified BP cuff system.

The hydrodynamic filter can be achieved using a sequential combination of resistive tubing and compliant tubing. The resistive tubing generates a flow resistance limiting the flow that can move across this element. The compliant tubing stores injected volume at a desired compliance rate. This hydraulic system generates the electrical equivalent of an RC low pass filter. In such a hydraulic system, the circuit may be understood as the time the compliant element needs to fill up through the resistive element.

In some implementations, the hydrodynamic filter comprises a resistive component and a compliant component. The resistive component is configured to impose a resistance to flow, thereby slowing down the flow, whereas the compliant component comprises a capacitive component configured to reduce pressure changes by storing air volume. In such embodiments, the compliant component may comprise tubing that connects the resistive component to the reference port. Additionally, the resistive component of the hydrodynamic filter may comprise rigid tubing with an internal diameter in the range of 10-200 μm. In some cases, an elasticity of the capacitive element is in the range of 0.2-2.0 MPa. The hydrodynamic filter may form an input connection to the reference port and thus regulate flow into the reference port. This configuration may provide the BP cuff system with steady pressure resulting in a smooth signal.

FIG. 11 shows an example of a BP cuff system having a passive configuration for the high accuracy sensor. As shown in FIG. 11, the system may include a BP cuff 1100. The system may also include pneumatic connections 106 for a high resolution pressure sensor 1114, a filter 1112 including a resistive component 1108 in series with a capacitive component 1110, and a reference port 1120. The system may also include an air pump 1102 and a BP monitor 1104. The air pump 1102 may be used to inflate the cuff 1100.

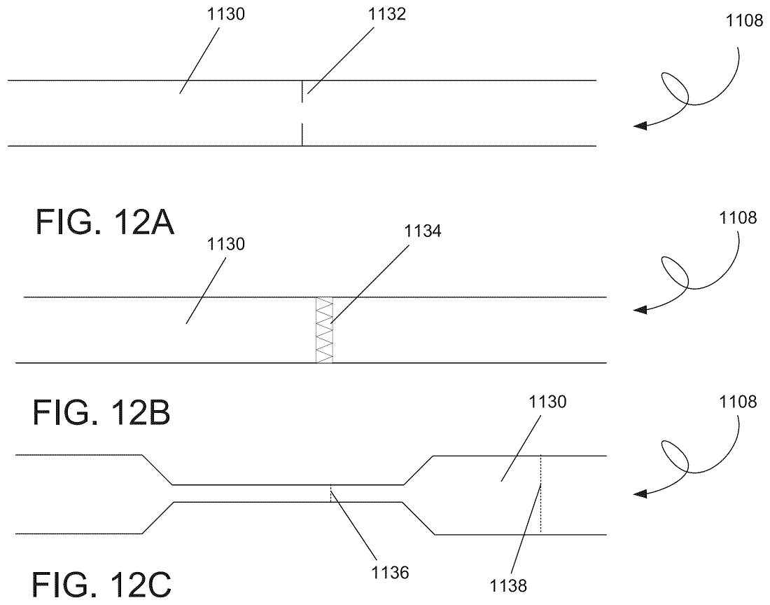

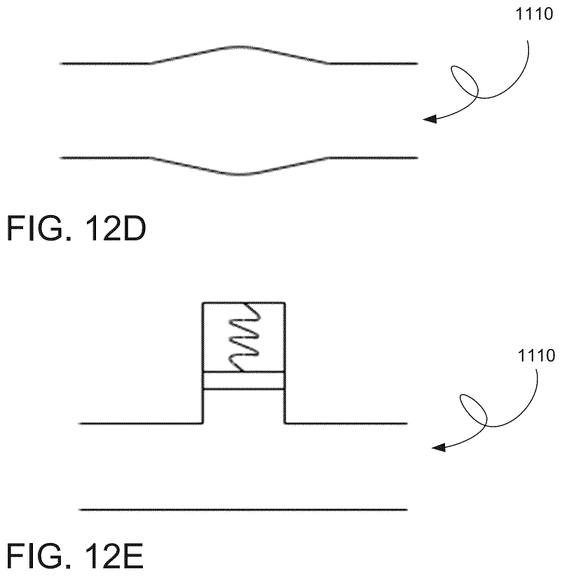

FIGS. 12A, 12B, and 12C are schematics of the resistive component 1108 comprising tubing 1130 with fixed orifice 1132, tube with inline filter 1134, and tube with ID 1136 smaller than ID 1138. As shown in FIG. 12A, the filter 1134 may comprise an orifice 1132. As shown in FIG. 12B, orifice 1132 may be configured in the tubing 1130 connecting to the reference port 1120. In further embodiments, the orifice may be adjustable. As shown in FIG. 12B, the tubing 1130 connecting to the reference port 1120 may be configured with an in-line filter 1134. As shown in FIG. 12C, the tubing 1130 connecting to the reference port 1120 may include a portion with a reduced diameter, or a small ID 1136 relative to the ID of the tubing 1138.

In some implementations, the filter 1134 may comprise a fixed or adjustable orifice 1132. The orifice 1132 may control the amount of air that can flow between the rest of the BP pressure cuff system and the reference port 1120. Air flow across the orifice 1132 is driven by a pressure differential. Limiting flow between compartments using an orifice instead of a valve results in smoothing out pressure oscillations while maintaining mean signal, acting as low pass filter on reference port side.

FIGS. 12D and 12E are schematics of the capacitive component 1110 including an elastic tube (FIG. 12D), and tube with a piston cylinder (FIG. 12E).

An important measurement may be the difference between the signal measured at the measurement port and at the reference port. Therefore, the output signal is the equivalent of a high pass filtered signal. Additionally, a self-adjusting reference port signal may also maintain a centered output signal. Using an orifice, as opposed to an air valve, allows the reference port to stay at the mean pressure signal. Maintaining the mean pressure signal is important to eliminate bias in the measured pulsations and maintain a centered signal with the high-resolution transducer.

In some implementations, the hydrodynamic filter may comprise a resistive component comprising a tube with a small ID. In such embodiments, the resistive component may comprise substantially rigid tubing with an internal diameter in the range of 10-200 μm. The ID of the tube may be significantly smaller in diameter than the ID of the tubing connected the rest of the BP pressure cuff system and reference port. The tubing with small ID effectively acts as a filter in which only the mean pressure is transmitted creating a flow dependent low pass filter to the reference port. Specific cutoff frequencies of the filter are designed using fluid dynamic principles and the characteristics of the measuring system and signal. The filtered signal will be dependent on the combination of volumetric flow rate and the material properties of the reference port side.

In some implementations, the system may be designed using a commercial arm cuff BP system. The system may be modified to include a plurality of pressure sensors with different operating ranges to measure and calibrate the pressure waveform with high accuracy. High resolution pressure sensors may be used for accurate signal measurement. Each high resolution pressure sensor may contain a measurement port and a reference port. High range pressure sensors may be used for absolute reference and signal calibration.

In some implementations, the system can be applied to any location in the body that has arteries close to the surface and can withstand a brief reduction or cessation of blood flow. Potential locations may include, but are not limited to, brachial, radial, femoral, and posterior tibial.

A pressure sensor may measure peripheral pressure pulse. Per fluid-solid interaction principles, the pressure at which cuff is inflated alters pressure-flow behavior in artery. Combinatorial waveform analysis may then be used to non-invasively assess cardiovascular health. A peripheral pulse waveform may be measured and then may be transformed to estimate the central waveform.

Pressure and flow velocity in closed system may be given by the Bernoulli equation (below). When a brachial cuff is inflated, the externally applied force alters the radius of the artery, ultimately changing the pressure flow proportion of the system. At a lower extreme, applying pressure to the artery below minimum DBP will cause no or minimal alteration to pressure flow behavior. At an upper extreme, pressure above the maximum SBP in the artery causes collapse of artery and cessation of blood flow. Any pressure between these two extremes may create a proportional alteration to the pressure flow relationship, again given by the Bernoulli equation. Comparing a measured waveform at two different hold pressures therefore allows derivation of the pressure-flow characteristics of system. Quantitative and qualitative comparisons of the waveforms may be performed. Plotting methods may also be used such as waveform versus time or waveform versus waveform.

Captured signals reflecting the pressure flow relationship in elastic arteries can be used to derive additional waveforms that may further characterize a patient. Fluid dynamic principles enable deriving waveforms, including, but not limited to, flow, velocity, and radial movement. Fluid-dynamics principles relate parameters such as pressure, velocity, forces, and volumes for static systems. Analyzing these systems with multiple measurement points allows to solve for the interrogated parameters. For example, velocity may be solved for by using DBP and sSBP hold pressure waveforms. The sSBP waveform completely obstructs flow, giving an absolute pressure reading. The DBP waveform represents a combination of pressure and flow. Therefore, the resulting flow may be measured during the DBP hold pressure. Similar derivations may be applied to other hold pressure combinations. The significance of the results obtained may depend on the underlying physics of the captured waveform(s).

Practical applications may require synchronization of waveforms. Solutions may include time synchronization of different waveforms using ECG or using known timing events during cardiac cycle including max dP/dt, start, and dicrotic notch. This cuff can have wired or wireless synchronization with other devices, such as Bluetooth or Wi-Fi with ECG.

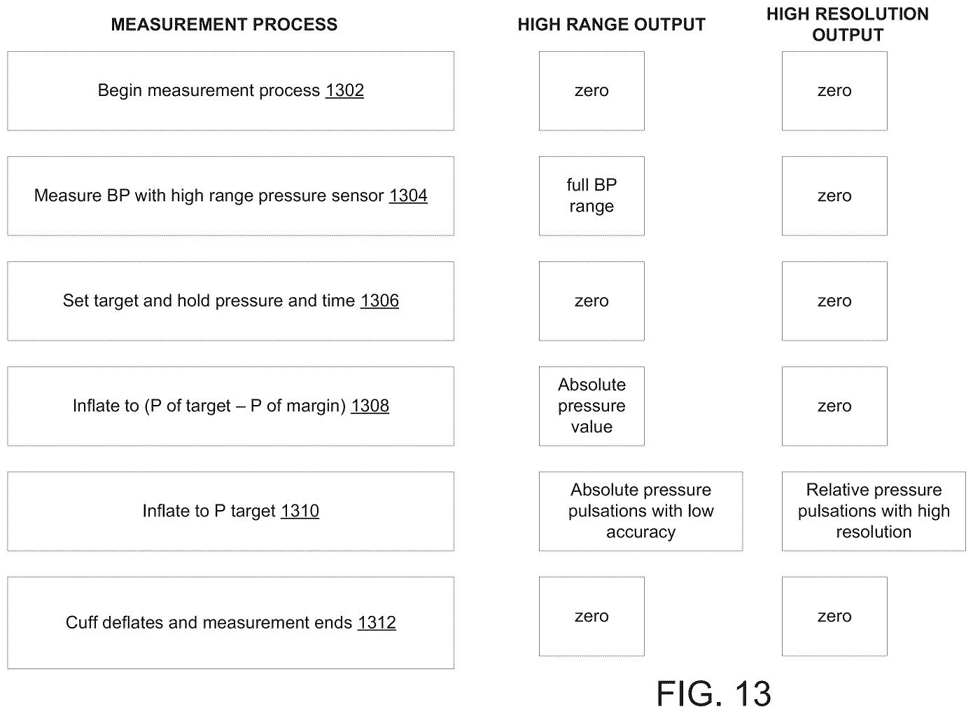

FIG. 13 shows an example of a non-invasive pulse pressure waveform measurement. A modified BP pressure cuff assembly, as described above, may be used to record pressure pulsations in the brachial artery. To perform the measurement a brachial BP cuff may be inflated around a patient's arm. A pump system may be used to inflate the cuff. A first operation 1302 may involve starting the measurement process. At the time of the first operation 1302, the output of a high range pressure sensor and a High resolution pressure sensor may both be zero. The pressure sensor may be used to measure the SBP and DBP of the patient. A second operation 1304 may involve measuring BP with a high range pressure sensor. At the time of the second operation 1304, the output of a high range sensor may the full BP range for the patient and the output of a high resolution pressure sensor may be zero.

As a third operation 1306, target and hold pressure and time may be set. For example, the cuff may be set to be inflated to a pressure of 100 mmHg and may be held for 40 seconds. Other pressures and timing are also possible. At the time of the third operation 1306, the output for both the high range and high resolution pressure sensors may be zero. Inflation pressure references and/or targets for the cuff may be obtained by performing traditional blood pressure arm cuff measurements. For example, hold pressures may be set at DBP, below DBP, at SBP, above SBP, and/or at MAP. Typical physiological ranges for these values are as follows: 40-120 mmHg for DBP; 50-150 mmHg for MAP; and 75-225 mmHg for SBP. Other pressures are also possible. For example, extremely sick subjects could have values outside of these ranges. For patient specific values, a specified pressure level may be employed to guide the hold pressure selection. To maintain the high resolution pressure sensor within its operating range, a pressure may applied to the reference port.

As a fourth operation 1308, the cuff may be inflated and held at a given pressure. For example, the cuff may be inflated to a pressure of P target, which may be one of the identified hold pressures. At the time of the fourth operation 1308, the high range sensor output may be the absolute pressure value and the high resolution pressure output may be zero. As a fifth operation 1310, the cuff may then be inflated to the target pressure. At the time of the fifth operation 1310, the high range sensor may have an output of the absolute pressure pulsations with low accuracy. The high resolution sensor may have an output of the relative pressure pulsations with high accuracy. As a sixth operation 1312, the cuff may deflate, ending the measurement period. At the time of the sixth operation 1312, each of the high and high resolution pressures sensors may have an output of zero. For multiple hold pressures, operations three to six may be repeated. Outputs from operation five may be combined for a calibrated high accuracy pulsation output.

In some implementations, comparisons of waveform captures may be performed using a low hold pressure and a high hold pressure above an upper pressure extreme. For instance, for the low hold pressure, pressure may be set at or just below DBP. For the high hold pressure, pressure may be at sSBP, cutting off the flow of blood. For example, pressure may be set at about SBP+35 mmHg. At the lower extreme, the waveform may represent a combination of static pressure and flow velocity. At the upper extreme, waveform only displays pressure characteristics. At sSBP hold pressures, cessation of flow in subclavian artery becomes closest waveform for representing static pressure read from a hole in the wall of the ascending aorta. This hold pressure allows for direct pressure waveform measurements in central arteries. Respective pressure waveform between sSBP and DBP can be plotted in pressure-pressure (“PP”) loop for cardiac health and disease assessment. This allows for creation of pressure-velocity (“PV”) loop applied for health assessments.

### Pressure-Velocity (“PV”) Loop Embodiment

As discussed above, flow at the brachial artery may be characterized with the Bernoulli equation for average flow, given by:

$${P}_{B} + \frac{1}{2} ρ {u}_{B}^{2} = {P}_{T}$$

where P_{B} is the pressure at the brachial artery, ρ is the fluid density, u_{b} is the flow velocity at the brachial artery, and P_{T} is the total pressure in the aortic arch.

At the sSBP pressure hold (P_{SS}), the brachial artery is completely occluded resulting in the u_{b}=0. Thus, the measured pressure at the cuff is the pressure in the aortic arch. As such:

P_{SS}=P_{B}=P_{T}

where P_{SS} is the sSBP hold pressure.

At the DBP pressure hold (P_{D}), the applanation condition measures the pressure in the brachial artery. The pressure in the brachial artery fits in the Bernoulli equation as shown below:

$${P}_{D} = {P}_{B} \to {P}_{B} + \frac{1}{2} ρ {u}_{B}^{2} = {P}_{T}$$

where P_{D} is the DBP hold pressure.

Equating the above through the total aortic arch pressure and solving for the velocity (u_{B}) gives:

$${P}_{SS} = {P}_{D} + \frac{1}{2} ρ {u}_{B}^{2} \\ {u}_{B} = \sqrt{\frac{2}{ρ} * ( {P}_{SS} - {P}_{D} )}$$

Plotting the sSBP pressure (P_{SS}) versus the DBP velocity (u_{D}) gives the (“PV”) loop. Data may be plotted and analyzed at any intermediate step. For example, SBP pressure versus DBP pressure may be analyzed.

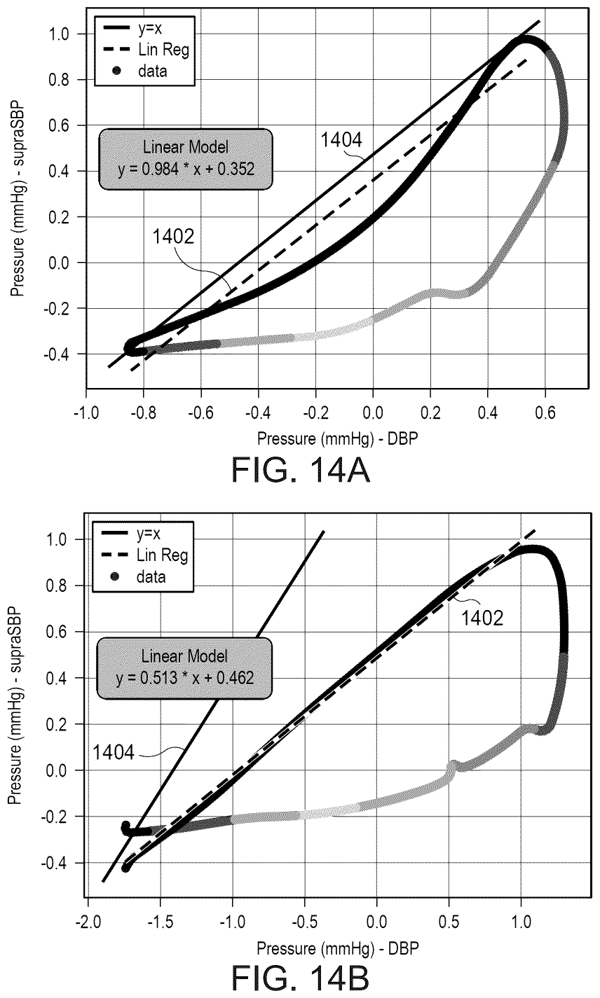

FIGS. 14A-14B shows examples of PV loop comparison of older (FIG. 14A) and younger (FIG. 14B) patients. Each of the PV loops have different features and shapes, Each includes a dashed line 1402 which models the slope of rising systolic portion. The slope can clearly differentiate patients by age, so it is a useful diagnostic. Each also includes a solid line 1404 which is a proportionality line. Other parameters include loop areas, curvatures, indentations, peak shifts, and other combinations of parameters.

Left Ventricular End-diastolic Pressure (“LVEDP”) Risk Prediction Embodiment

LVEDP is an important clinical measurement used to predict, diagnose, and/or assess the risk for heart failure. Currently, the threshold for heart failure is an LVEDP measurement of about 18 mmHg. Because LVEDP is a valuable diagnostic measurement, non-invasive methods for measuring LVEDP may allow clinicians to predict risk of heart failure earlier and with accuracy.

A non-invasive pulse waveform analysis and classification algorithm may be used to form an LVEDP risk prediction.

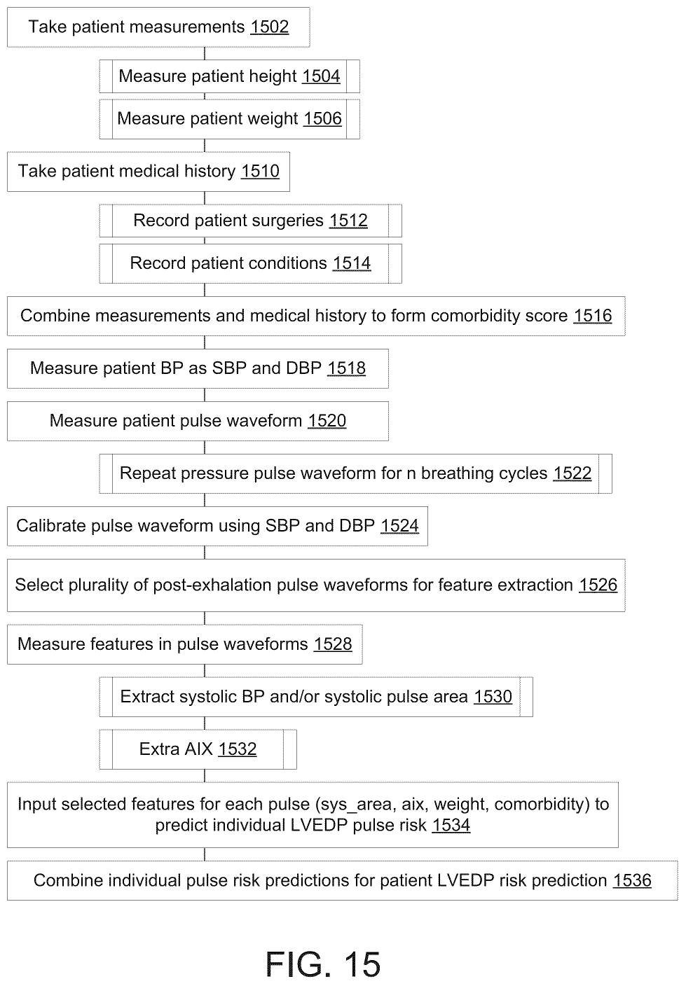

FIG. 15 shows an example of a non-invasive LVEDP risk prediction method 1500 involving several operations. A first operation 1502 may involve taking patient measurements. A sub operation 1504 of the first operation 1502 may involve measuring the patient's height. A sub operation 1506 of the first operation 1502 may involve measuring the patient's weight. Other patient measurements may also be taken. A second operation 1508 may involve taking a patient's medical history. A sub operation 1510 to the second operation 1508 may involve recording patient surgeries. Patient surgeries, such as heart surgeries, may be recorded. Other medical information, such as procedures and other treatments may also be recorded. A sub operation 1512 to the second operation 1508 may involve recording patient conditions. Patient conditions may include known instances of cardiovascular conditions such as heart failure, myocardial infarction, and cardiomyopathy. Other heart conditions, comorbid conditions, and/or general health conditions may be recorded. Other patient information may also be recorded such as genetic predispositions, family history, lifestyle factors and other information.

A third operation 1516 may involve combining recorded patient measurements and medical history to form a comorbidity score.

A fourth operation 1518 may involve measuring the patient's BP as SBP and DBP using a commercially available and/or conventional brachial cuff or some other measurement means.

A fifth operation 1520 may involve measuring the patient's pulse waveforms. The pulse waveforms may be measured using a modified BP cuff in accordance with the foregoing embodiments. For example, a modified blood pressure cuff that inflates to specific pressures and holds those pressures to capture a waveform at the set pressure may be used. Hold pressures may include DBP, SBP, MAP, and/or sSBP. In some embodiments, an sSBP, which completely cuts off the flow of blood through an artery, may be used. The inflation pressure may be, for example, about 100 mmHg. The hold time may be about, for example, 40 seconds.

Sub operation 1522 to the fifth operation 1520 may involve performing the pulse waveform measurements for a duration long enough to account for pressure amplitude fluctuations throughout a breathing cycle. Measured amplitudes may be highest at the post-exhalation stage of the breathing cycle.

A sixth operation 1524 may include calibrating the measured pulse waveform(s) using the SBP and DBP measurements. The waveform may be calibrated with pressure units utilizing BP measurement results. Calibration methods may include the methods disclosed in the following section of this disclosure.

A seventh operation 1526 may include selecting a plurality of post-exhalation waveforms for feature extraction. Post-exhalation waveforms may be selected because these waveforms may track the highest LVEDP reading throughout a breathing cycle. Extracted features and/or parameters of interest may include augmentation index (“AIX”), systolic pulse area, and/or systolic BP. Other desirable and/or relevant features and/or parameters may also be extracted.

An eighth operation 1528 may include measuring and/or extracting the features and/or parameters of interest in the pulse waveforms. A sub operation 1530 to the eight operation 1528 may involve extracting SBP or systolic pulse area. A sub operation 1532 to the eighth operation 1528 may involve extracting the AIX.

In some embodiments, a classification algorithm may be used to assess risk. Inputs for the algorithm may be pulse features of systolic pulse area, augmentation index and patient features of weight and comorbidity score. Using the inputs and algorithm, a probability of having LVEDP greater than or equal to a failure threshold may be generated. In an embodiment, the failure threshold may be set at about 18 mmHg. In another embodiment, the threshold may be set at about 15 mmHg or at another value of clinical relevance selected when training the algorithm. This process may be repeated for post-exhalation pulses in n breathing cycles to generate n probability predictions. A plurality of measurements may be taken. For example, in an embodiment, two or three measurement may be taken. The probability of the plurality of individual pulses may be combined into a single risk prediction using ensemble methodologies. The predictions may be processed together to generate a single LVEDP risk prediction. Ensemble methodologies may include averaging the probabilities and/or may include more complex methods of aggregating the probabilities.

Accordingly, a ninth operation 1534 may involve inputting selected features and/or parameters for each pulse to predict individual LVEDP risk. The selected features and/or parameters may include the systolic pulse area, AIX, patient weight, and comorbidity score. Other parameters and/or combinations of parameters are also possible. Likewise, a tenth operation 1536 may involve combining individual pulse risk predictions for patient LVEDP risk prediction.

### Calibration Embodiment

BP cuff measurements may serve as useful clinical measurements because peripheral BP, as measured via a cuff, tends to track central BP in healthy patients. Unfortunately, in patients experiencing cardiovascular issues, the relationship between peripheral BP and central BP may degrade. The severity of the cardiovascular issues in a patient may affect the extent to which the peripheral-central BP relationship degrades. However, because BP cuff measurements are quick, non-invasive, and inexpensive to perform, BP cuff measurements remain an important diagnostic tool for patients experiencing cardiovascular issues. Though the peripheral-central BP relationship degrades in such patients, calibration methods may allow a peripheral BP measurement performed with, for example, a brachial BP cuff, to serve as a proxy for a central BP measurement even in a patient experiencing severe issues.

A modified BP cuff system, such as systems described in the foregoing embodiments, may be used to measure peripheral BP. Peripheral BP may be measured over several breathing holds cycles due to pressure fluctuations caused by breathing. A non-invasive pulse signal measured using a modified BP cuff system may be calibrated to track central BP magnitudes in a patient.

An envelope function may be used to correct a peripheral BP measurement and calibrate a signal. The envelope function may comprise a relationship between pulse amplitude and cuff pressure at measurement site. The measurement cite may be an artery. An envelope function may be constructed by measuring pulse amplitude corresponding to cuff pressure across multiple breath holds.

A calibration method may include several operations. A first operation may involve measuring peripheral BP in the form of SBP, DBP, and MAP using a conventional and/or commercially available oscillometric cuff. These measured values may not be accurate. Specifically, these measured values may not track measurement taken in vivo due to amplitude fluctuations caused by breathing and/or due to other errors.

A second operation may involve using a modified BP cuff. The modified BP cuff may be a modified BP system including high resolution pressure sensors and high range pressure sensors, as described in the foregoing embodiments. The second operation may involve inflating the modified BP cuff to a set pressure value. Pulsations may be recorded at that pressure value. The BP cuff may be inflated over a range of set pressure values. Pulsations may be recorded for each pressure value. The second operation may involve taking operations over multiple breath holds to account for pressure fluctuations caused by breathing. For each breath hold, the measured waveform may be analyzed to compare signal pulse amplitude to the set pressure of the modified BP cuff. Measurements may be taken over a plurality of holds to reconstruct a proxy of the envelope function. For example, measurements may be taken over two, three, or more breath holds.

A third operation may involve using the measurements taken during the second operation to calculate parameters to correct for pressure fluctuations due to breathing changes and ultimately to calibrate peripheral measurements to central measurements. This may be accomplished by using an envelope function to derive the parameters needed to correct the peripheral measurements.

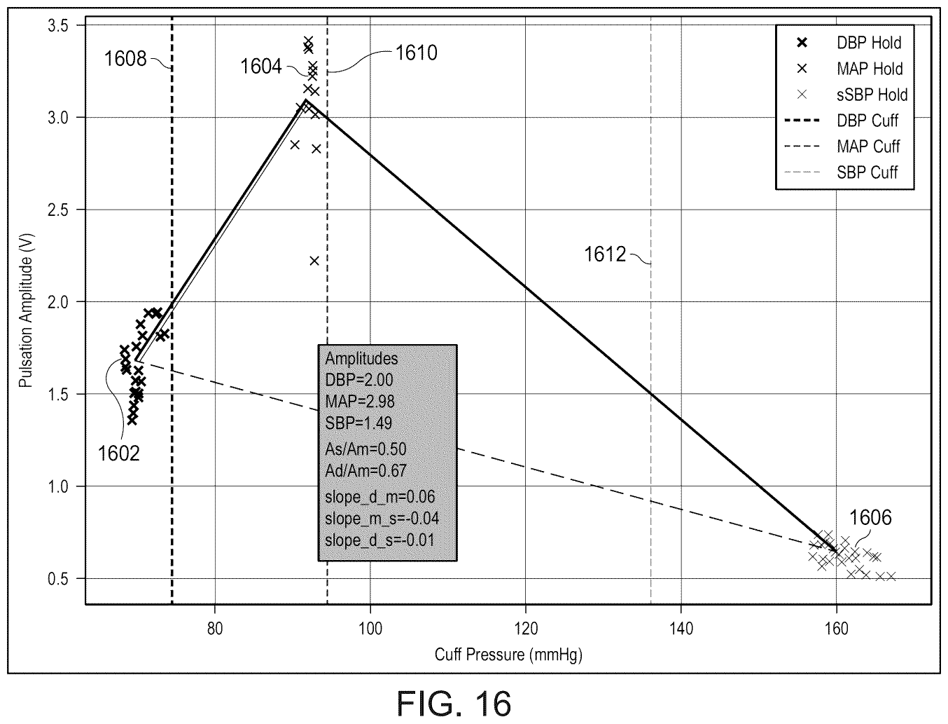

FIG. 16 shows an example of an envelope function reconstructed with a BP measurement and three pressure holds. The three pressure holds are DBP, MAP, and sSBP. For the DBP pressure hold, the modified BP cuff was inflated with minimal pressure, allowing blood to flow through the artery substantially unobstructed. For the MAP pressure hold, the modified BP cuff was inflated to MAP. For the sSBP hold, the modified pressure cuff was inflated above the pressure for SBP to effectively cut off any blood flow through the artery. Cuff pressure is shown in FIG. 16 as dashed vertical lines for the three hold values. The three holds values include the DBP hold 1608, the MAP hold 1610, and the sSBP hold 1612.

FIG. 16 also shows individual record pulsations measured at each hold pressure. FIG. 16 shows individual pulsations 1602 for the DBP hold, individual pulsations 1604 for the MAP hold, and individual pulsations 1606 for the sSBP hold. As shown in the example of FIG. 16, multiple pulsations may be recorded for each pressure hold. The pulse amplitudes of the individual pulsations 1602, 1604, 1606 may be measured with a pressure sensor. In the example shown in FIG. 16, pulsation amplitude is reported in volts (V). These pulse amplitude measurements may also be converted into other pressure units.

As discussed above, breathing may cause substantial fluctuations in central BP. The voltage-based signals from a cuff pressure hold may show breathing fluctuations just as in a catheter aortic signal. Therefore, BP values reported by a modified BP cuff measurement may be assumed to be mean values. Pulse signals may be calibrated to pressure units by adjusting SBP and DBP values over a breath pattern and correctly scaling the measured pressure signal. For each individual pulsation in a segment of pulsations, the pulse amplitude difference from the mean pulse amplitude of the segment may be used to correct the SBP and/or DBP values for breathing fluctuations.

For example, a model to correct DBP values from peripheral to central DBP using the envelope function derived parameters is given by:

$${DBP}_{corr} = {DBP}_{cuff} + {m}_{1} * \frac{{A}_{d}}{{A}_{m}} + {m}_{2} * {MAP}_{cuff} + b$$

where A_{d}/Aₘ is the ratio between the pulse amplitude at DBP versus MAP and DBP_{cuff} and MAP_{cuff} are the DBP and MAP reported by the cuff BP reading respectively, and m₁, m₂, and b are coefficients optimized for the correlation.

SBP values may be corrected using forward and reflect wave peaks measurable in a pulse waveform signal. For the brachial cuff, a potential hold pressure that shows these features is sSBP. Corrected SBP may be given by:

$${SBP}_{corr} = {SBP}_{cuff} + {m}_{1} * ( {P}_{1} - {P}_{2} ) + b$$

where SBP_{corr} is the corrected SBP value to track central BP, SBP_{cuff} is the SBP cuff measurement readout, P₁ is the peak pressure of the first peak in systole, P₂ is the peak pressure of the second peak in systole, and m₁ and b are coefficients optimized for the correlation.

A linear envelope function model may be used to calculate the actual pressure as shown in the following close form equation:

$${P}_{adj} = {P}_{calib} + \frac{Δ P A}{{slope}_{p}}$$

where P_{adj} is the breathing adjusted pressure, P_{calib} is the BP reported value, ΔPA is the pulse amplitude difference from mean, and slopeₚ is the envelope function slope for the specific pressure (slope_{DBP} or slope_{SBP}). Pressure may be either SBP or DBP.

In an uncalibrated segment of pulsations, repeating the foregoing calculations for every pulsation in the segment of pulsations and utilizing signal scaling methodologies, all pulsations can be calibrated with SBP and DBP values that reflect breathing patterns. The model presented assumes a linear relationship between measurement points and a fixed envelope function for a given subject. With more hold pressures, a more detailed envelope function may be reconstructed and may increase model accuracy. Calibration may be applied to SBP and/or DBP independently.

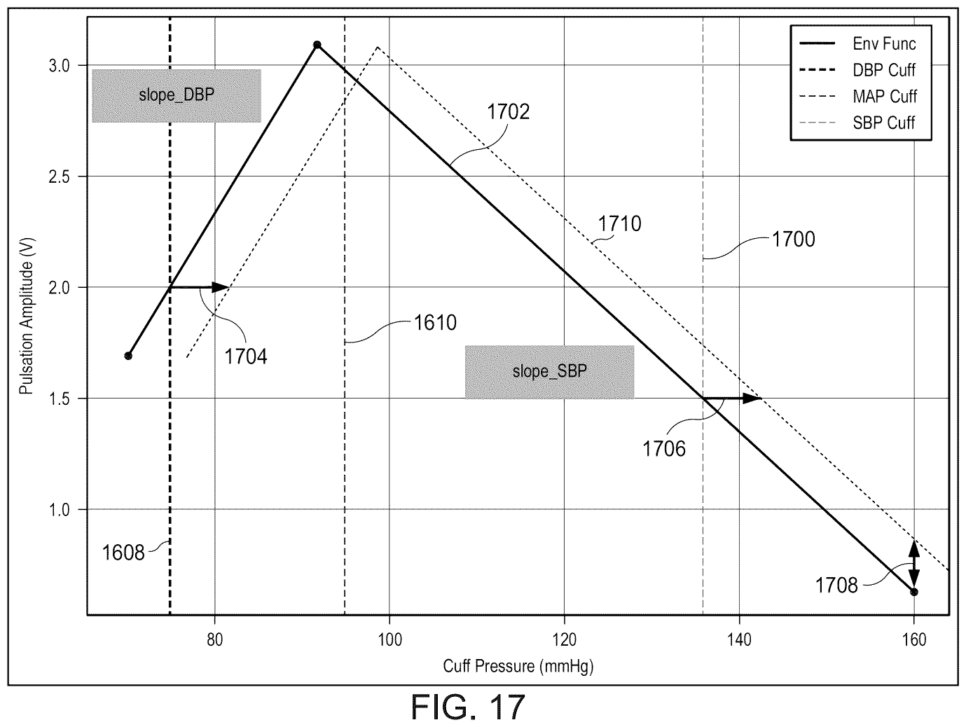

FIG. 17 shows an example of an envelope function used to estimate SBP and DBP changes following pulse amplitude fluctuations. As in FIG. 16, pressure cuff holds are represented by dashed vertical lines. FIG. 17 shows a DBP pressure cuff hold value 1608, a MAP pressure cuff hold value 1610, and a SBP pressure cuff hold value 1700. The arrow 1708 at the bottom right of FIG. 17 shows the pulse amplitude deviation from mean pulse amplitude. The arrow 1706 above and right of the arrow 1708 shows the SBP increase. The arrow 1704 at the left of FIG. 17 shows the DBP increase. The envelope function 1702 can then be used to estimate SBP and DBP changes following fluctuations in pulse amplitude, which may be caused by breathing. FIG. 17 shows the estimate 1710. FIG. 17 is exemplary only. The methodology described above with reference to FIG. 17 may be performed with different devices, threshold conditions, and quantities if the necessary information described is obtained.

In some embodiments, a calibration method incorporating breathing fluctuations may also serve as a diagnostic tool in cardiology. For example, the condition of pulsus paradoxus is defined as a fall in SBP greater than about 10 mmHg during inspiration. This condition may be observed during cardiac tamponade or right ventricle distension such as in severe acute asthma or chronic obstructive pulmonary disease. Therefore, an embodiment of a calibration method may involve setting a threshold of about 10 mmHg.

In this document, the terms “machine readable medium,” “computer readable medium,” and similar terms are used to generally refer to non-transitory mediums, volatile or non-volatile, that store data and/or instructions that cause a machine to operate in a specific fashion. Common forms of machine readable media include, for example, a hard disk, solid state drive, magnetic tape, or any other magnetic data storage medium, an optical disc or any other optical data storage medium, any physical medium with patterns of holes, a RAM, a PROM, EPROM, a FLASH-EPROM, NVRAM, any other memory chip or cartridge, and networked versions of the same.

These and other various forms of computer readable media may be involved in carrying one or more sequences of one or more instructions to a processing device for execution. Such instructions embodied on the medium, are generally referred to as “instructions” or “code.” Instructions may be grouped in the form of computer programs or other groupings. When executed, such instructions may enable a processing device to perform features or functions of the present application as discussed herein.

In this document, a “processing device” may be implemented as a single processor that performs processing operations or a combination of specialized and/or general-purpose processors that perform processing operations. A processing device may include a CPU, GPU, APU, DSP, FPGA, ASIC, SOC, and/or other processing circuitry.

The terms “substantially” and “about” used throughout this disclosure, including the claims, are used to describe and account for small fluctuations, such as due to variations in processing. For example, they can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

To the extent applicable, the terms “first,” “second,” “third,” etc. herein are merely employed to show the respective objects described by these terms as separate entities and are not meant to connote a sense of chronological order, unless stated explicitly otherwise herein.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

It should be appreciated that all combinations of the foregoing concepts (provided such concepts are not mutually inconsistent) are contemplated as being part of the inventive subject matter disclosed herein. In particular, all combinations of claimed subject matter appearing in this disclosure are contemplated as being part of the inventive subject matter disclosed herein.

What is claimed is:

1. A method, comprising:
obtaining a time-domain representation of a first pulse pressure waveform signal of a brachial artery of a subject;
converting the time-domain representation of the first pulse pressure waveform signal to a frequency-domain representation of the first pulse pressure waveform signal;
selecting a first plurality of frequency components of the frequency-domain representation of the first pulse pressure waveform signal;
predicting, using a non-linear mapping, based on the first plurality of frequency components, a second plurality of frequency components of a frequency-domain representation of a second pulse pressure waveform signal of an ascending aorta or a left ventricle (LV) of the subject; and
converting the frequency-domain representation of the second pulse pressure waveform signal to a time-domain representation of the second pulse pressure waveform signal.

2. The method of claim 1, wherein predicting the second plurality of frequency components comprises predicting each of the second plurality of frequency components using at least some of the first plurality of frequency components as an input to the non-linear mapping.

3. The method of claim 1, wherein a number of the first plurality of frequency components is different from a number of the second plurality of frequency components.

4. The method of claim 1, wherein:
converting the time-domain representation of the first pulse pressure waveform signal to the frequency-domain representation of the first pulse pressure waveform signal comprises: applying a fast Fourier transform (FFT) to the time-domain representation of the first pulse pressure waveform signal;
selecting the first plurality of frequency components of the frequency-domain representation of the first pulse pressure waveform signal comprises selecting a first number of Fourier harmonic modes of the frequency-domain representation of the first pulse pressure waveform signal; and
converting the frequency-domain representation of the second pulse pressure waveform signal to the time-domain representation of the second pulse pressure waveform signal comprises: applying an inverse FFT to the frequency-domain representation of the second pulse pressure waveform signal.

5. The method of claim 4, wherein:
the non-linear mapping comprises a regressor; and
predicting the second plurality of frequency components comprises: applying the regressor to the first number of Fourier harmonic modes to obtain a second number of Fourier harmonic modes of the frequency-domain representation of the second pulse pressure waveform signal.

6. The method of claim 5, wherein the first number of Fourier harmonic modes is 5 or more, and the second number of Fourier harmonic modes is 10 or more.

7. The method of claim 1, wherein:
the non-linear mapping comprises a trained model; and
predicting the second plurality of frequency components comprises: predicting, using the trained model, given at least the first plurality of frequency components as an input, the second plurality of frequency components.

8. The method of claim 7, wherein:
the method further comprises calculating one or more waveform parameters corresponding to the time-domain representation of the first pulse pressure waveform signal; and
predicting the second plurality of frequency components comprises: predicting, using the trained model, given at least the first plurality of frequency components and the one or more waveform parameters as inputs, the second plurality of frequency components.

9. The method of claim 7, wherein:
the method further comprises obtaining one or more physiological parameters of the subject, the one or more physiological parameters comprising an age, weight, height, or gender of the subject; and
predicting the second plurality of frequency components comprises: predicting, using the trained model, given at least the first plurality of frequency components and the one or more physiological parameters as inputs, the second plurality of frequency components.

10. The method of claim 1, wherein obtaining the time-domain representation of the first pulse pressure waveform signal comprises:
performing, using a blood pressure (BP) cuff of a BP cuff system, a blood pressure measurement of the subject to obtain one or more blood pressure values corresponding to the subject;
inflating, based on the one or more blood pressure values corresponding to the subject, the BP cuff to a subject specific pressure value; and
capturing, using the BP cuff system, while the BP cuff is inflated to the subject specific pressure value, the first pulse pressure waveform signal associated with the brachial artery of the subject.

11. The method of claim 10, wherein:
the one or more blood pressure values comprise a systolic blood pressure (SBP) of the subject; and
the subject specific pressure value comprises a supra systolic blood pressure (sSBP) greater than the SBP.

12. The method of claim 10, wherein:
the first pulse pressure waveform signal corresponds to a single cardiac cycle; and
capturing the first pulse pressure waveform signal comprises:
capturing, using the BP cuff system, while the BP cuff is inflated to the subject specific pressure value, a third pulse pressure waveform signal corresponding to more than one cardiac cycle; and
isolating the first pulse pressure waveform signal from the third pulse pressure waveform signal.

13. The method of claim 1, further comprising: applying pulse waveform analysis to the time-domain representation of the second pulse pressure waveform signal to assess a cardiovascular health condition of the subject.

14. The method of claim 13, wherein:
the second pulse pressure waveform signal corresponds to a central aortic pressure of the subject; and
applying pulse waveform analysis to the time-domain representation of the second pulse pressure waveform signal to assess the cardiovascular health condition of the subject comprises:
estimating, based one or more parameters of the time-domain representation of the second pulse pressure waveform signal, an arterial stiffness or a subendocardial viability ratio.

15. The method of claim 13, wherein:
the second pulse pressure waveform signal corresponds to a left ventricular pressure of the subject; and
applying pulse waveform analysis to the time-domain representation of the second pulse pressure waveform signal to assess the cardiovascular health condition of the subject comprises: estimating, based one or more parameters of the time-domain representation of the second pulse pressure waveform signal, a ventricular contractility or left ventricular end diastolic pressure.

16. The method of claim 1, wherein the second pulse pressure waveform signal is of the LV of the subject, and the method further comprises:
obtaining an LV pressure curve of the subject associated with the time-domain representation of the second pulse pressure waveform signal;
capturing, using an imaging device, one or more images of the LV of the subject;
measuring, using the one or more images of the LV, an LV volume curve of the subject; and
obtaining, using the LV pressure curve and the LV volume curve, an LV pressure-volume curve.

17. A non-transitory computer-readable medium having executable instructions stored thereon that, when executed by a processor, cause the processor to perform operations comprising:
obtaining a time-domain representation of a first pulse pressure waveform signal of a brachial artery of a subject;
converting the time-domain representation of the first pulse pressure waveform signal to a frequency-domain representation of the first pulse pressure waveform signal;
selecting a first plurality of frequency components of the frequency-domain representation of the first pulse pressure waveform signal;
predicting, using a non-linear mapping, based on the first plurality of frequency components, a second plurality of frequency components of a frequency-domain representation of a second pulse pressure waveform signal of an ascending aorta or a left ventricle of the subject; and
converting the frequency-domain representation of the second pulse pressure waveform signal to a time-domain representation of the second pulse pressure waveform signal.

18. A method, comprising:
obtaining, using a brachial cuff, a time-domain representation of a first pulse pressure waveform signal of a brachial artery of a subject;
reconstructing, using a frequency-domain representation of the first pulse pressure waveform signal, a frequency-domain representation of a second pulse pressure waveform signal of a left ventricle (LV) of the subject;
obtaining, using the frequency-domain representation of the second pulse pressure waveform signal of the LV of the subject, an LV pressure curve of the subject in the time domain;
capturing, using an imaging device, one or more images of the LV of the subject;
measuring, using the one or more images of the LV, an LV volume curve of the subject; and
obtaining, using the LV pressure curve and the LV volume curve, an LV pressure-volume curve.

19. The method of claim 18, wherein:
obtaining the time-domain representation of the first pulse pressure waveform signal of the brachial artery of the subject comprises: capturing, using the brachial cuff, multiple blood pressure measurements of the subject; and
the multiple blood pressure measurements and the one or more images of the LV are synchronously captured.

20. The method of claim 18, wherein:
the method further comprises capturing an electrocardiogram (ECG) of the subject; and
obtaining the LV pressure-volume curve comprises synchronizing, using the ECG, the LV pressure curve and the LV volume curve.
